(19) United States
(12) Patent Application Publication
(10) Pub. No.: US 2018/0028612 A1
(43) Pub. Date: Feb. 1, 2018

(54) Compositions and methods of using a soluble TNF-alpha receptor modified for increased half-life

(71) Applicant: DNX BIOTECH, LLC, San Diego, CA (US)

(72) Inventors: Rajiv Datar, Irvine, CA (US); Carl K. Edwards, III, San Marcos, CA (US)

(21) Appl. No.: 15/662,434

(22) Filed: Jul. 28, 2017

Related U.S. Application Data
(63) Continuation of application No. PCT/US2015/067055, filed on Dec. 21, 2015.
(60) Provisional application No. 62/108,825, filed on Jan. 28, 2015.

(51) Int. Cl. A61K 38/19; A61K 47/26; A61K 47/60; A61K 38/17

(52) U.S. Cl. CPC A61K 38/191; A61K 38/1712; A61K 47/26; A61K 47/60; C07K 14/525

(57) ABSTRACT

Methods and pharmaceutical compositions for preventing and/or treating acute and chronic inflammation and autoimmune diseases are provided herein. Tumor necrosis factor-α (TNFα) promotes an inflammatory response, which causes clinical problems associated with inflammation and autoimmune disorders such as rheumatoid arthritis, ankylosing spondylitis, inflammatory bowel disease, psoriasis, hidradenitis suppurativa, and refractory asthma. TNFα is also implicated in promoting pathogenesis of diabetic retinopathy leading to loss of retinal microvascular cells. Methods herein contain the step of administering a prophylactic and/or therapeutic formulation of a pharmaceutical composition containing a recombinant soluble human TNF receptor or portions thereof which are TNFα inhibitors. These pharmaceutic compositions have been modified by conjugating natural amino acids such as proline and alanine, and/or serine (PA/S) via PASylation® to create a linear polypeptide that possesses fewer of the processing, preparation, formulation, cost, and other long-term issues of administering PEGylated drugs.

### CROSS-REFERENCE TO RELATED APPLICATIONS

This application claims the benefit of U.S. provisional application 62/108,825 filed Jan. 28, 2015, which is hereby incorporated by reference in its entirety.

### FIELD OF THE INVENTION

This invention relates to half-life extended forms of biopharmaceutical compositions for use in the effective, safe, and convenient treatment of metabolic and immunological diseases. Half-life modification and drug delivery technologies are shown herein that improve efficacy, safety, and patient compliance factors for the administration of effective and safe treatments of chronic inflammation and autoimmune disease such as diabetic retinopathy and arthritis. Improvements in these factors reduce the cost and clinical burden associated with present treatments.

### BACKGROUND OF THE INVENTION

Two distinct human Tumor Necrosis Factor-α (TNFα) receptors have been identified: the 55-kd or p55 receptor type I (TNF-RI), and the 75-kd or p75 receptor type II (TNF-RII). TNF-RI and TNF-RII exist in both cell-surface and soluble forms, and bind TNF with different affinities. TNFα cell-surface receptors are present on most cell types, including macrophages, lymphocytes, and neutrophils. TNFα must bind to two or three cell-surface receptor molecules for signal transduction to occur. Numerous biological effects of TNFα are mediated by intracellular signaling of the high-affinity TNF-RI receptor. Monomeric fragments that contain the extracellular portion of the cell-surface receptors are naturally occurring forms due to proteolytic cleavage, and are referred to as soluble TNF receptors. References cited herein are hereby incorporated by reference in their entireties.

Since the discovery of tumor necrosis factor TNF-alpha (TNFα) about 30 years ago, more than 20 additional proteins that signal through over 30 receptors have been identified as a members of the TNF Receptor Superfamily (TNFRSF). These cytokine receptors are characterized by the ability to bind tumor necrosis factors (TNFs) with an extracellular cysteine-rich domain. With the exception of nerve growth factor (NGF), TNFs are homologous to the archetypal TNFα. Members of the superfamily have a wide tissue distribution, and play important roles ranging from regulation of the normal biological processes such as immune responses, hematopoiesis, and morphogenesis to their role in pathologies such as tumorigenesis, transplant rejection, septic shock, viral replication, bone resorption, and autoimmunity. Thus, many approaches to harness the potency of TNF superfamily members to treat human diseases have been developed. TNF and TNF agonistic molecules have been approved for human use in several countries worldwide. Many other TNF family members show promise for therapeutic applications for cancer, infectious disease, transplantation and autoimmunity.¹ The term TNF receptor is often used to refer to the archetypal members of the superfamily, namely TNF-RI and TNF-R2, which recognize TNFα.

TNFα is a pleiotropic cytokine implicated for early inflammatory changes seen in the diabetic retina. In a diabetic retina, astrocytes and Muller cells are potential sources of TNFα′. In addition, TNFα is found in the extracellular matrix, endothelium, and vessel walls of fibrovascular tissue and is elevated in the vitreous of eyes with diabetic retinopathy^{2,3}. In rat models, diabetes of two weeks duration increased TNFα level by greater than two-fold⁴. In humans, TNFα immunoreactivity is seen in the majority of retinal specimens obtained from patients with diabetic retinopathy′. A first generation TNF inhibitor etanercept (Enbrel®) has been shown to reduce intercellular adhesion molecule 1 levels, endothelial nitric oxide synthase gene expression, and nuclear factor kappa B (NF-κB) activity in a diabetic retina¹. Research shows that diabetes-enhanced levels of TNF plays a prominent role in microvascular cell death in both type 1 and type 2 diabetic retinas. These data show a potential therapeutic benefit of inhibiting TNFα activity in preventing the progression of early diabetic retinopathy, for which there is currently no effective preventive treatment.

Since excess TNFα activity is associated with disease pathogenesis particularly in inflammatory conditions, there is a need for TNFα antagonists and methods for their use in the treatment of inflammatory diseases. Concerns have been raised regarding the side effects of currently approved protein-based TNFα antagonists, including Enbrel®, after initial dosing of once per week in RA patients, the dosing of Enbrel® has to be increased by 3-fold to about 150 mg, and the frequency of dosing must be increased from an initial once per week to two to three times per week. Additional concerns include exacerbation of latent tuberculosis, worsening of congestive heart failure, and increased risk of lymphoma⁴⁰. Furthermore, there are patients who either become severely recalcitrant, or simply do not respond to currently approved TNFα antagonists. Therefore, there is a continuing need to identify additional TNFα antagonists.

Soluble TNF receptors (sTNF-Rs) are commonly found in vertebrate organisms. Increased concentrations of sTNF-Rs have been found in the circulation of patients with rheumatoid arthritis (RA). sTNF-R concentrations are higher in synovial fluid samples compared with plasma sample concentrations in patients with RA. Signal transduction occurs when TNFα binds to and dimerizes two or three receptors of either the TNF-RI protein or TNF-RII protein on the cell surface. Naturally occurring TNFα inhibitors, containing 4 domains (4.0D) or truncated forms thereof, e.g., three domains (3.0D) and 2.6 domains (2.6D), of the extracellular region of TNF-RI, are referred to as TNF binding proteins (TNFbp) or soluble TNF receptor (sTNF-R). These molecules have been found in the tissue, serum, synovial fluid, and synovial explant cultures obtained from patients with active RA. The presence of sTNF-RI has been shown to correlate with RA disease activity.

A wide array of biological agents have been designed and commercialized to inhibit TNFα^{5,6}. Examples include: (1) TNFα type II soluble receptor fusion protein (e.g., etanercept, Enbrel®, Amgen, Inc.); (2) anti-human TNFα chimeric (mouse×human) monoclonal antibody (mAb) (e.g., infliximab, Remicade®, Centocor Ortho Biotech, Inc); (3) fully humanized mAb, (e.g., adalimumab, Humira®, Abbvie Inc.); (4) a human mAb (e.g., golimumab, Simponi®, Centocor Ortho Biotech, Inc.), and (5) PEG (polyethylene glycol)-ylated Fab fragment anti-TNFα antibody (certolizumab pegol, Cimzia®, UCB Pharma SA). A biosimilar version of inflixamab, CTP-13 (e.g., humanized chimeric inflixamab biosimilar IgG₁κ mAB, Rensima®, Celltrion Healthcare Inc.) has been approved in South Korea.

There have been other anti-TNFα product candidates shown to be active in human clinical trials. A TNFα type I soluble receptor (p55) fusion protein (lenercept, Roche), demonstrated short-term efficacy in European and North American phase 2 clinical trials in patients with RA, but was shown to be highly immunogenic upon longer-term dosing. The hinge region joining the full-length p55 receptor to the Fc region of the fusion protein appears to contain several antigenic epitopes responsible for the immunogenicity³. Anti-lenercept antibodies were bound to Fc receptors but were not detectable to sTNF-RI and had neither neutralizing nor antagonistic properties⁷⁻⁹. TNFbp, a dimeric PEGylated form of the full-length sTNF-RI produced in E. coli, has been observed preclinically¹⁰ and in clinical trials¹¹ to be active as a TNFα inhibitor. The immunogenicity of TNFbp reduced the clearance rate of the molecule and reduced the serum half-life in a phase I/II clinical trial. TNFbp was observed to be unsuitable for a chronic indication¹¹. However, proof of concept was demonstrated by a decrease in swollen and tender joint counts over a 21-day period¹². Marked reduction (45% to 60%) in swollen joint counts was seen after intravenous (IV) doses of 100 μg/kg and 300 μg/kg¹¹.

A recombinant C-terminal truncated form of the human soluble tumor necrosis factor receptor type I (sTNF-RI) was produced in E. coli¹³. This soluble receptor contains the first 2.6 of the 4 domains of the intact sTNF-RI protein. A monoPEGylated form of this protein was produced using a 30 kD methoxyPEG aldehyde (PEGsunercepe) with about 85% selectivity for the N-terminal amino group. This protein was shown to be less immunogenic in primates than the 4.0 domain protein or other versions of E. coli-derived sTNF-RI which were either PEGylated at different sites or with different molecular weight PEGs. The reason for the increased immunogenicity of the third and fourth domains of the native sTNF-RI has not been fully determined, and anecdotal evidence show that refolding during the purification process was a major issue¹⁴. The 30 kD PEG sTNF-RI also has a longer serum half-life compared to sTNF-RI modified by lower molecular weight PEGs. PEG polymers are used to increase the viscosity of the formulated drug product. This protein reduces the inflammatory response in a number of RA animal models. In addition, clinical trial phase I/II and early clinical trial phase II data in humans shows that PEG-sTNF-RI is non-immunogenic and that weekly dosing with this drug reduces the number of tender and swollen joints in RA patients. PEG-sTNF-RI was shown to have comparable American College of Rheumatology efficacy scores to alternative anti-TNFα molecules currently used to treat RA patients¹⁵. Development of PEG sTNF-RI by Amgen, Inc. appears to have been halted circa 2005 after successful clinical phase IIc trials, and Amgen has neither publicly announced new studies regarding this program nor has a commercial product emerged since that time.

Tumor necrosis factor-binding protein, TBP-1 (onercept, Serono), is a soluble glycoprotein corresponding to the extracellular portion of the human TNF-RI^{16,17}. This soluble receptor, which contains the 4 domain binding region to TNFα and TNFβ, is naturally shed by enzymatic cleavage from the cell membrane into circulation. TBP-1 is excreted into the urine where it was first identified and characterized¹⁸. Cloning of the receptor mRNA permitted the manufacture of recombinant human TBP-1 (rhTBP-1) by genetic engineering in mammalian cells (Chinese hamster ovary cells). rhTBP-1 is a 20-kD molecular weight glycoprotein reproducing the identical amino acid sequence and glycosylation pattern to the natural form as characterized in urine^{19,20}. By specifically binding to the bioactive trimeric form of both TNFα and TNFβ, rhTBP-1 neutralizes their bioactivities. Preclinical studies both in vitro and in vivo in animal disease models as well as toxicology studies have shown the activity and safety of TBP-1.

Truncated sTNF-Rs are chemically modified in vitro with a host of water-soluble, non-biological, synthetic polymers, to create a multitude of chemically-derivatized truncated sTNF-Rs. See, U.S. Pat. No. 6,989,147. The best known of these synthetic, non-biodegradable polymers is PEG. U.S. Pat. No. 6,989,147 shows that the average molecular weight of the PEG polymer is preferably between about 5 kDa and about 50 kDa, more preferably between about 12 kDa and about 40 kDa, and most preferably between about 20 kDa and about 40 kDa.

The current predominant half-life extension technology of PEGylation, which was developed in the early 1990s, is associated with the following issues: high cost-of-goods; post-production chemical coupling and processing steps leading to additional product losses; often, considerably lowered biological activity of the drug payload; high viscosities; and increasing evidence of accumulation in organs such as renal tubule cells, macrophages, choroid plexus epithelial cells, leading to problems of vacuolation²¹. The clinical development of various PEGylated products such as PEGsunercept®, PEGylated αIL1β Fab, GlycoPEGylated factor Vila among others, have either been terminated or suspended.

Generally, the higher the molecular weight of the PEG and/or the more branches of the PEG polymer coupled to the protein of interest, the higher the polymer:protein ratio. The higher the polymer:protein ratio, the higher the viscosity of the chemically-coupled product, which is a creates difficulties related to the ease-of-injection and mode-of-delivery factors. U.S. Pat. No. 7,700,722 shows that proteins chemically conjugated to PEG polymers having a molecular weight in the range of 20 kDa to 35 kDa and viscosities of up to 400 cP. At these viscosities, not only are injection times long (i.e. about 80 seconds or more), but significantly thicker gauge needles must be used (i.e., about 23 G) than needles used for lower viscosity composition, which makes for extremely painful injections.

sTNFR-I and sTNFR-II are members of the nerve growth factor/TNF receptor superfamily of receptors which includes the nerve growth factor receptor (NGF), the B cell antigen CD40, 4-1BB, the rat T-cell antigen MRC OX40, the Fas antigen, and the CD27 and CD30 antigens²². The most conserved feature among this group of cell surface receptors is the cysteine-rich extracellular ligand binding domain, which occur in four repeating motifs of about forty amino acids and which contains four to six cysteine residues at positions which are well conserved²².

Recombinantly-produced TNF inhibitors have been taught in the art. For example, European patent (EP) 393438 and EP 422339 show the amino acid and nucleic acid sequences of a mature, recombinant human 30 kDa TNF inhibitor (also known as a p55 receptor and as sTNF-RI) and a mature, recombinant human 40 kDa inhibitor (also known as a p75 receptor and as sTNF-RII) as well as modified forms thereof, e.g., fragments, functional derivatives, and variants. EP 393438 and EP 422339 also show methods for isolating the genes responsible for coding the inhibitors, cloning the gene in suitable vectors and cell types, and expressing the gene to produce the inhibitors. Mature recombinant human 30 kDa TNF inhibitor and mature recombinant human 40 kDa TNF inhibitor have previously been demonstrated to be capable of inhibiting TNF (See, EP 393438 and EP 422339).

EP 393438 shows a 40 kDa TNF inhibitor Δ51 and a 40 kDa TNF inhibitor Δ53, which are truncated versions of the full-length recombinant 40 kDa TNF inhibitor protein having 51 or 53 amino acid residues, respectively, at the carboxyl terminus of the mature protein, removed. Accordingly, a skilled artisan would appreciate that the fourth domain of each of the 30 kDa TNF inhibitor and the 40 kDa inhibitor is not necessary for TNF inhibition. Domain-deleted, truncated derivatives of the 30 kDa and 40 kDa TNF inhibitors have been generated. The truncated derivatives without the fourth domain retain full TNF binding activity, while those derivatives without the first, second, or third domain, do not retain TNF binding activity^{23,24,25}.

Half-life extension technologies have been developed such as the polypeptide-based, random-coil domain (RCD) technology called PASylation®²⁶⁻²⁹. See, Skerra et al., WO 2011/144756 published Nov. 24, 2011 and Skerra et al., WO 2008/155134 published Dec. 24, 2011, which are hereby incorporated by reference in their entireties. The polypeptides of PASylation® contain sequences of amino acids proline, alanine, and optionally serine (PA/S, or PAS to indicate that serine is present) residues. The polymer which is a combination of amino acid residues results in cancellation of the distinct secondary structure preferences of each amino acid residue to form a stably disordered polypeptide. Biologically active proteins attached to at least one PAS polypeptide, which contains a domain with an amino acid sequence that assumes a random coil conformation, have been observed to have increased in vivo and/or in vitro stability compared to the protein in its native state lacking this adduct.

### BRIEF SUMMARY OF THE INVENTION

Various embodiments of the invention herein relate to a composition for preventing or treating a subject for at least one of an inflammation, an autoimmune disease, and a metabolic disease, the composition including a full-length or a truncated form of a receptor protein that is a member of the superfamily of sTNF receptors (sTNF-Rs); and an adduct covalently linked to the receptor protein that increases the half-life of the composition in the subject, and the composition having decreased immunogenicity than the full-length or the truncated form of the receptor protein alone, or than a corresponding PEGylated form of the protein.

In certain embodiments of the invention, the receptor protein is at least one selected from the TNF receptor superfamily of: sTNF-RI, sTNF-RII, death receptor 6 (DR6), cluster of differentiation 95 (CD95), decoy receptor 3 (DcR3), death receptor 3 (DR3), tumor necrosis factor receptor superfamily member 12A (Fn14), death receptor (DR4), death receptor (DR5), decoy receptor 1 (DcR1), decoy receptor 2 (DcR2), osteoprotegerin (OPG), receptor activator of nuclear factor κ B (RANK), herpesvirus entry mediator (HVEM), lymphotoxin-β receptor (LTβR), glucocorticoid-induced TNFR-related protein (GITR), cluster of differentiation 40 (CD40), cluster of differentiation 30 (CD30), cluster of differentiation 27 (CD27), tumor necrosis factor receptor superfamily member 4 (OX40), tumor necrosis factor receptor superfamily member 9 (41BB), nerve growth factor receptor (NGFR), B-cell maturation antigen (BCMA), transmembrane activator and CAML interactor (TACI), BAFF receptor 3 (BR3), x-linked ectodermal dysplasia receptor (XEDAR), ectodysplasin A receptor (EDAR), tumor necrosis factor receptor superfamily member 19 (TROY), and tumor necrosis factor receptor superfamily member 19L (RELT). For example, the receptor protein is at least one selected from the group of a p55 TNFα monomeric receptor (sTNF-RI) protein; a p75 TNFα monomeric receptor (sTNF-RII) protein; and the truncated form of the receptor protein including at least one of: domain 1 or a portion thereof, domain 2 or a portion thereof, domain 3 or a portion thereof, and domain 4 or a portion thereof.

In certain embodiments of the invention, the composition is biodegradable in vivo in the subject. In an aspect of the invention, the composition is biodegradable by kidney enzymes of the subject. In certain embodiments, the adduct of the composition is a polypeptide containing proline and alanine, and/or serine (PA/S or PAS if serine is present) or is naturally occurring sugars containing heparosan molecules. In certain embodiments, the adduct of the composition is a linear polypeptide chain containing at least one of natural amino acid residues or a combination of natural and unnatural amino acid residues. In certain embodiments, the adduct of the composition increases the half-life of the proteins at least about 10-fold. In certain embodiments, the adduct increases the half-life of the proteins by a factor of at least about 300-fold. In certain embodiments, the adduct of the composition is a PAS polypeptide that forms a monodisperse mixture as determined using mass spectroscopy. In certain embodiments, the adduct of the composition is covalently linked at the C-terminus of the sTNF-RI protein or the sTNF-RII protein, or at the N-terminus of the sTNF-RI protein or the sTNF-RII protein.

In certain embodiments, the adduct of the composition is a plurality of adducts, and a first adduct is covalently linked at the N-terminus and a second adduct is covalently linked at the C-terminus of the sTNF-RI protein or the sTNF-RII protein. In certain embodiments, the adduct of the composition is covalently linked to the sTNF-RI protein or the sTNF-RII protein at a position internal to the N-terminus and the C-terminus. In certain embodiments, the adduct is a plurality of adducts, each at a different position in the sTNF-RI protein or the sTNF-RII protein. In certain embodiments, the adduct of the composition is a plurality of adducts, and each of the plurality is covalently linked to a different domain of the sTNF-RI protein or the sTNF-RII protein, and the domains are at least one selected from the group of domains of the full-length form of the sTNF-RI protein or the sTNF-RII protein containing domains 1, 2, 3, and 4. In certain embodiments, the adduct of the composition further includes at least one selected from the group of drugs consisting of: an anti-inflammatory drug, a steroidal drug, and a non-steroidal drug. For example, the anti-inflammatory drug is methotrexate.

In certain embodiments, the adduct of the composition is located at an immunogenic site of the sTNF-RI protein or of the sTNF-RII protein and masks the immunogenicity. In certain embodiments, the adduct of the composition is at least about 200 amino acid residues. For example, the adduct is at least about 1200 amino acid residues. In certain embodiments, the half-life of the composition in vivo is at least about 25 hours, at least about 75 hours, at least about 125 hours, at least about 175 hours, at least about 225 hours, or at least about 275 hours. In certain embodiments, the truncated form of sTNF-RI protein includes an amino acid sequence consisting of at least one sequence selected from the group consisting of: SEQ ID NO: 3, SEQ ID NO: 4, SEQ ID NO: 5, SEQ ID NO: 6, SEQ ID NO: 7, and SEQ ID NO: 8. In certain embodiments, the adduct of the composition is not PEG.

Various embodiments of the invention herein relate to a method of preventing or treating a subject for at least one of an inflammation or an autoimmune disease, the method including: engineering a composition containing a truncated form of p55 TNFα monomeric receptor (sTNF-RI) or of p75 TNFα monomeric receptor (sTNF-RII) proteins, and the sTNF-RI or the sTNF-RII proteins containing an adduct that increases the half-life of the proteins in the subject, and the sTNF-RI or the sTNF-RII proteins containing the adduct is less immunogenic than a PEGylated sTNF-RI or sTNF-RII protein; and administering the composition to the subject.

In certain embodiments, the method further includes, prior to administering, the step of formulating the sTNF-RI or the sTNF-RII proteins in a form that is effective for a prophylactic or a therapeutic use. In certain embodiments, the method further includes, prior to administering, the step of genetically conjugating the adduct to the sTNF-RI or to the sTNF-RII proteins. In certain embodiments, the method further includes, prior to administering, the step of chemically conjugating the adduct to the sTNF-RI or to the sTNF-RII proteins. In certain embodiments, the method further includes, prior to administering, increasing the half-life of the sTNF-RI protein or the sTNF-RII protein by conjugating a PAS polypeptide or naturally occurring sugars containing heparosan molecules to the proteins.

Various embodiments of the invention herein relate to a composition for preventing or treating a subject for at least one of an inflammation, an autoimmune disease, and a metabolic disease, the composition including: a truncated form of p55 TNFα monomeric receptor (sTNF-RI) protein, the truncated form of sTNF-RI protein containing the amino acid sequence consisting of SEQ ID NO: 6; and a PAS polypeptide covalently linked to the protein that increases the half-life of the composition in the subject, the PAS polypeptide having a length of at least about 600 amino acid residues. For example, the half-life of the composition is within a range of about 200 hours to about 250 hours.

An embodiment of the present invention provides functionally active, truncated sTNFRs, modified for increased half-lives using a molecular biology approach, rather than using post-production chemical coupling methods and technologies.

### BRIEF DESCRIPTION OF THE DRAWINGS

FIG. 1 is a diagram of the problem and the optimal solution to half-life extension and the effective delivery of biopharmaceutical drugs.

FIG. 2 is a graph of de-convoluted zero-charge mass spectra showing highly polydisperse nature of PEG residues for increasing the half-lives of biopharmaceutical compositions. See, Bagal et al., Anal. Chem., 80: 2408-2418 (2008).

FIG. 3 is a drawing of the structure formed by natural amino acids or by a combination of natural and unnatural amino acids having specific length “n” creating a linear polypeptide.

FIG. 4 is a drawing of the structure formed by sugar molecules containing heparosan of multiple repeat units “n”.

FIG. 5 is a graph of mass spectroscopy data of the size distribution and highly monodisperse nature of the polypeptide structure of FIG. 3.

FIG. 6 is a graph that compares the viscosities between various lengths of amino acid residues exemplified by the repeating structure of FIG. 3 and PEG polymers in the preferred molecular weight range.

FIG. 7 is a diagram of the TNF super family ligands and the known receptors of each.¹

FIG. 8 are x-ray crystallography results depicting the three-dimensional structure of biologically active forms, 13, of recombinant soluble human p55 or p75 soluble TNF receptors. See, Naismith et al., Structure, 4(11): 1251-1262 (1996); Banner et al., Cell, 73: 431-445 (1993).

FIG. 9 is a drawing of the process of formation of a biopharmaceutical composition and variants with extended half-lives, from a combination of the proteins of FIG. 7, with the PAS polypeptide, 10, of FIG. 3 and its variants. The PAS polypeptide, 10, may be at either end of the biopharmaceutical composition variant, at one end only, or at both ends as illustrated in FIG. 8, or within the protein variant.

FIG. 10 shows the formation of a biopharmaceutical composition and its variants with extended half-lives, by combining the proteins of FIG. 7 with heparosan molecules of FIG. 4 and its variants. Heparosan molecules, 11, may be at either end of the biopharmaceutical composition, at one end only, or at both ends as illustrated in FIG. 9, or within the protein.

FIG. 11 is a drawing of a biopharmaceutical composition with an extended half-life containing at least one protein of FIG. 7 in combination the PAS polypeptide 10 of FIG. 3 and its variants and/or heparosan molecules, 11, of FIG. 4 and its variants. The PAS polypeptide, 10, or heparosan molecules, 11, may be individually and independently located at either end of the protein, at one end only, at both ends, or within the protein.

FIG. 12 is a drawing showing an increase in the effective molecular volume of a biologically active form, 13, of sTNF-Rs as shown in FIG. 7, when suitably attached to a PAS polypeptide, 10, of FIG. 3 because of the picosecond to femtosecond vibrations of the PAS polypeptide, 10.

FIG. 13 is a drawing of the increase in the hydrodynamic volume of the biologically active forms of sTNF-RI protein and sTNF-RII protein of FIG. 7 conjugated with different variants of either the PAS polypeptide, 10, of FIG. 3 and/or with heparosan molecules, 11, of FIG. 4.

FIG. 14 is a plot of elimination half-life versus body weight using the principles of interspecies allometric scaling for the structures shown in FIG. 8 and its variants across several clinically-relevant species.

FIG. 15A is the nucleic acid sequence of the full-length form of the sTNF-RI protein (SEQ ID NO: 1). The shaded portion of the nucleic acid sequence is the 2.6D variant of the full-length sTNF-RI protein (SEQ ID NO: 2).

FIG. 15B is the amino acid sequence (SEQ ID NO: 3) that corresponds with the nucleic acid sequence of FIG. 15A.

FIG. 15C is the amino acid sequence at amino acid position 41 to amino acid position 201 of the full-length sTNF-RI protein (SEQ ID NO: 3) containing 4.0 domains (4.0D) (SEQ ID NO: 4).

FIG. 15D is the amino acid sequence at amino acid position 41 to amino acid position 167 of the full-length sTNF-RI protein (SEQ ID NO: 3) containing 3.0 domains (3.0D) (SEQ ID NO: 5) of.

FIG. 15E is the amino acid sequence at amino acid position 41 to amino acid position 148 of the full-length sTNF-RI protein (SEQ ID NO: 3) containing 2.6 domains (2.6D) (SEQ ID NO: 6).

FIG. 15F is the amino acid sequence at amino acid position 49 to amino acid position 148 of the full-length sTNF-RI protein (SEQ ID NO: 3) containing 2.3 domains (2.3D) (SEQ ID NO: 7).

FIG. 16 is the amino acid sequence of the full-length form of the sTNF-RII protein (SEQ ID NO: 8).

FIG. 17A-FIG. 17C are illustrations of cloning strategies/construct and plasmid map for genetically fusing a PAS polypeptide sequence to sTNF-Rs for expression in prokaryotic cells such as E. coli or in mammalian cells such as Chinese Hamster Ovary (CHO) cells.

### DETAILED DESCRIPTION OF THE INVENTION

Products currently on the market cause problems with immunogenicity; rapid clearance from the human body; viscosity; and routes, methods, and frequency of administration. References cited herein are hereby incorporated by reference in their entireties.

PASylation® provides advantages that PEGylation cannot: it maintains high target affinity; it has not elicited immunogenicity in preclinical trials to date; it is biodegradable such that it is efficiently degraded by kidney enzymes; and it is stable in the blood stream. The PAS polypeptide shows no polydispersity; and does not require in vitro coupling steps, thereby not negatively affecting the cost of goods factor. The PAS polypeptide has lower viscosity for the comparable molecular weight of PEG; and, the half-life extension is tunable from 10-fold to greater than 300-fold. These advantages render the protein modified by PASylation® more efficacious, safer, and considerably more convenient by way of lowered dosing and frequency of administration bringing about an increase in patient compliance.

There are concerns with using existing drugs in many patients who are immunosuppressed. These drugs are not modified, which results in rapid clearance of the drug from the body, and in turn has to be compensated by higher quantities and/or by more frequent dosing regimens, leading to an increase in clinical burden.

Certain embodiments of the invention herein mask the immuno-suppressive nature of a biopharmaceutical drug and increase its half-life in the body. Consequently, the drug is not rejected by the body, and does not result in immune reactions leading to lower quantities or frequency of dosing.

Certain embodiments of the invention herein modify one or more of the molecules cited above to improve the therapeutic outcomes to patients suffering from life-long diseases such as diabetic retinopathy and arthritis.

sTNF-RI and sTNF-RII are reduced in size to either exclude or include specific domains and retain biological activity²³⁻²⁵. Certain embodiments of the invention herein are based on the discovery that truncated or full-length forms of sTNF-RI and sTNF-RII genetically fused to polypeptide chains via PASylation® retain biologic activity with reduced antigenicity and greatly increased half-lives. These molecules have one less potentially destabilizing deamidation site and have fewer disulfide bridges. PASylation® simplifies the process of refolding and purifying, and PASylated molecules have a reduced number of sites for potential antigenic epitopes.

Techniques such as mutagenesis for replacing, inserting, or deleting one or more selected amino acid residues are well known to one skilled in the art (e.g., U.S. Pat. No. 4,518,584). Typically there are two principal variables in the construction of each amino acid sequence variant: location of the mutation site and nature of the mutation. In designing each variant, the location of each mutation site and the nature of the mutation depended on the biochemical characteristic(s) to be modified. Each mutation site was modified individually or in series by: (1) substituting first with conservative amino acid choices and then with more radical selections, depending on results, (2) deleting the target amino acid residue, or (3) inserting amino acid residues adjacent to the site. These techniques were used to make deletions, insertions, and substitutions in the amino acid sequence of sTNF-Rs to create a variety of truncated forms that remained biologically active.

An embodiment of the invention herein contemplates sTNF-Rs containing genetically-fused PASylated moieties that do not exhibit the viscosity-related drawbacks of the current art as exemplified by the process of PEGylation®.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, 22^{nd} Ed.; Gennaro, Mack Publishing, Easton, Pa. (2012) provides various carriers used in formulating pharmaceutical compositions and known techniques for the preparation thereof. Examples of materials which serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants may also be present in the composition, the choice of agents and non-irritating concentrations to be determined according to the judgment of the formulator.

### Therapeutically Effective Dose

Compositions, according to the method of the present invention, may be administered using any amount and by any route of administration effective for preventing or treating a subject for an inflammation or an autoimmune disease. An effective amount refers to a sufficient amount of the composition to beneficially prevent or ameliorate the symptoms of the disease or condition.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., liver function, cancer progression, and/or intermediate or advanced stage of macular degeneration; age, weight and gender of the patient; diet, time and frequency of administration; route of administration; drug combinations; reaction sensitivities; level of immunosuppression; and tolerance/response to therapy. Long acting pharmaceutical compositions might be administered hourly, twice hourly, every three to four hours, daily, twice daily, every three to four days, every week, or once every two weeks depending on half-life and clearance rate of the particular composition.

The active agents of the pharmaceutical compositions of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration and total dosing range and route of administration. Such information is used to determine useful doses and routes for administration in humans.

A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). The dose ratio of toxic to therapeutic effects is the therapeutic index, which is expressed as the ratio, LD₅₀/ED₅₀. Pharmaceutical compositions which exhibit large therapeutic indices are preferred. The data obtained from cell culture assays and animal studies are used in formulating a range of dosage for human use.

### Administration of Pharmaceutical Compositions

As formulated with an appropriate pharmaceutically acceptable carrier in a desired dosage, the pharmaceutical composition or methods provided herein is administered to humans and other mammals for example topically for skin tumors (such as by powders, ointments, creams, or drops), orally, rectally, mucosally, sublingually, parenterally, intracisternally, intravaginally, intraperitoneally, intravenously, subcutaneously, bucally, sublingually, ocularly, or intranasally, depending on preventive or therapeutic objectives and the severity and nature of the cancer-related disorder or condition.

Injections of the pharmaceutical composition include intravenous, subcutaneous, intra-muscular, intraperitoneal, or intra-ocular injection into the inflamed or diseased area directly, for example, for esophageal, breast, brain, head and neck, and prostate inflammation.

Liquid dosage forms are, for example but not limited to, intravenous, ocular, mucosal, pharmaceutically acceptable emulsions, microemulsions, solutions, suspensions, syrups and elixirs. In addition to at least one active agent, the liquid dosage forms may contain inert diluents commonly used in the art such as, for example, water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, and fatty acid esters of sorbitan, and mixtures thereof. Besides inert diluents, the ocular, oral, or other systemically-delivered compositions also include adjuvants such as wetting agents, and emulsifying and suspending agents.

Dosage forms for topical or transdermal administration of the pharmaceutical composition herein including ointments, pastes, creams, lotions, gels, powders, solutions, sprays, inhalants, or patches. The active agent is admixed under sterile conditions with a pharmaceutically acceptable carrier and preservatives or buffers may be required. For example, ocular or cutaneous routes of administration are achieved with aqueous drops, a mist, an emulsion, or a cream. Administration is in a therapeutic or prophylactic form. Certain embodiments of the invention herein contain implantation devices, surgical devices, or products which contain disclosed compositions (e.g., gauze bandages or strips), and methods of making or using such devices or products. These devices may be coated with, impregnated with, bonded to or otherwise treated with the composition described herein.

Transdermal patches have the added advantage of providing controlled delivery of the active ingredients to the eye and body. Such dosage forms can be made by dissolving or dispensing the compound in the proper medium. Absorption enhancers are used to increase the flux of the compound across the skin. The rate can be controlled by either providing a rate controlling membrane or by dispersing the compound in a polymer matrix or gel.

Injectable preparations of the pharmaceutical composition, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions which are dissolved or dispersed in sterile water or other sterile injectable medium. Slowing absorption of the agent from subcutaneous or intratumoral injection was observed to prolong the effect of an active agent. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release is controlled. Examples of other biodegradable polymers include poly (orthoesters) and poly(anhydrides). Depot injectable formulations are also prepared by entrapping the agent in liposomes or microemulsions which are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate or dicalcium phosphate and/or fillers or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents such as, for example, cetyl alcohol, and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that releases the active agent(s) only, preferably in a certain part of the intestinal tract, and optionally in a delayed manner. Examples of embedding compositions include polymeric substances and waxes.

### Recombinant Expression and Preparation of the Fusion Polynucleotides

Nucleic acid sequences encoding truncated sTNF-Rs are readily obtainable in a variety of ways including, without limitation, chemical synthesis, cDNA or genomic library screening, expression library screening, and/or polymerase chain reaction (PCR) amplification of cDNA. These methods and others, which are useful for isolating such nucleic acid sequences are set forth in Sambrook et al.³⁰; by Ausubel et al.³¹; and Berger and Kimmel³².

Chemical synthesis of nucleic acid sequences which encode truncated sTNFRs were accomplished using methods well known in the art. See, Engels et al.³³ and Wells et al.³⁴. Alternatively, a suitable technique for obtaining a nucleic acid sequence is PCR. In this method, cDNA is prepared from poly(A)+RNA or total RNA using the enzyme reverse transcriptase. Two primers, typically complementary to two separate regions of cDNA (oligonucleotides) encoding a truncated sTNFR are added to the cDNA along with a polymerase such as Taq polymerase. Polymerase amplifies the cDNA region between the two primers.

Another technique for obtaining a nucleic acid sequence is screening a cDNA library or a genomic library (a library prepared from total genomic DNA). The source of the cDNA library is typically at least one tissue from a species that is believed to express the desired protein in reasonable quantities. The source of the genomic library may be any tissue or tissues from any mammalian or other species believed to harbor a gene encoding a form of truncated sTNFR.

The present invention relates to nucleic acid molecules encoding the biologically-active, half-life extended, truncated forms of sTNF-Rs as described herein. Accordingly, the nucleic acid molecule contained a nucleic acid sequence encoding a truncated form of a biologically active sTNF-R and a nucleic acid sequence encoding an amino acid sequence, which forms and/or adopts either entirely or in part, a random coil conformation domain (RCD), and confers the desired half-life extension characteristics under specific physiological conditions. Preferably, the nucleic acid molecule is in a vector.

Cells were transfected with the nucleic acid molecule or vectors as described herein. The nucleic acid molecules were fused to suitable expression control sequences to ensure proper transcription and translation of the polypeptide as well as signal sequences to ensure cellular secretion or targeting to organelles. Such vectors may contain further genes such as marker genes which allow for the selection of said vector in a suitable host cell and under suitable conditions.

Preferably, the nucleic acid molecule is in a recombinant vector in which the nucleic acid molecule encoding the herein described biologically-active, half-life extended, truncated sTNF-R(s) protein is operatively linked to expression control sequences allowing expression in prokaryotic or eukaryotic cells. Expression of the nucleic acid molecule encompasses transcription of the nucleic acid molecule into a translatable mRNA. Regulatory elements permitting expression in prokaryotic host cells include: lambda PL, lac, trp, tac, tet, or T7 promoter in E. coli. Potential regulatory elements ensuring expression in eukaryotic cells, preferably mammalian cells or yeast, are well known to those of ordinary skill in the art. Regulatory sequences ensure initiation of transcription, and optional poly-A signals ensure termination of transcription and stabilization of the transcript. Additional regulatory elements include transcriptional as well as translational enhancers, and/or naturally-associated or heterologous promoter regions. Examples of regulatory elements for expression in eukaryotic host cells are the AOX1 or GAL1 promoter in yeast or the CMV, SV40, RSV promoter (Rous sarcoma virus), CMV enhancer, SV40 enhancer, or a globin intron in mammalian and other animal cells. Apart from elements that are responsible for the initiation of transcription, such regulatory elements also contain transcription termination signals, such as the SV40-poly-A site or the tk-poly-A site, downstream of the coding region²⁸.

Methods which are well known to those of ordinary skill in the art were used to construct recombinant vectors. See, Sambrook et al.³⁰ and Ausubel et al.³¹. Examples of suitable expression vectors are Okayama-Berg cDNA expression vector pcDV1 (Pharmacia), pCDM8, pRc/CMV, pcDNA1, pcDNA3, pPICZalpha A (Invitrogen), or pSPORT1 (GIBCO BRL). Furthermore, depending on the expression system, leader sequences capable of directing the polypeptide to a cellular compartment or secreting it into culture medium are added to the coding sequence of the nucleic acid molecule of the invention.

The compositions are in solid or liquid form and are, inter alia, a powder, a tablet, a solution, an aerosol, a nanoparticle, or attached to a nanoparticle. The medicament of the invention contained further biologically active agents, depending on the intended use of the pharmaceutical composition.

The pharmaceutical compositions are administered in any of several different routes, e.g., by parenteral, subcutaneous, intraperitoneal, topical, intra-bronchial, intra-pulmonary, and intra-nasal administration and, if desired for local treatment, intra-lesional administration. Parenteral administrations include intra-peritoneal, intra-muscular, intra-dermal, subcutaneous, intra-venous, or intra-arterial administration. The compositions are also administered directly to the target site, e.g., biolistic delivery to an external or internal target site, such as an affected organ.

Examples of suitable pharmaceutical carriers, excipients and/or diluents are well known in the art and include phosphate buffered saline solutions, water, emulsions, such as oil/water emulsions, various types of wetting agents, and sterile solutions, etc. Compositions containing such carriers were formulated by well-known conventional methods. Carriers contain material which, when combined with the biologically active protein, retains the biological activity of the biologically active protein (see Remington's Pharmaceutical Sciences)³⁵. Preparations for parenteral administration include sterile aqueous or non-aqueous solutions, suspensions, and emulsions. Examples of non-aqueous solvents are propylene glycol, P, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present including, for example, anti-microbials, anti-oxidants, chelating agents, inert gases, and the like. The pharmaceutical composition herein contains proteinaceous carriers, like, e.g., serum albumin or immunoglobulin, preferably of human origin.

These pharmaceutical compositions were administered to the subject at a suitable dose. The dosage regimen was determined by the attending physician and clinical factors. As is well known in the medical arts, dosages for any one patient depend upon many factors, including the patient's size, body surface area, age, particular compound to be administered, sex, time and route of administration, general health, and other drugs being administered concurrently. Pharmaceutically active composition were administer in amounts between 1 μg and 20 mg/kg body weight per dose, e.g. between 0.1 mg to 10 mg/kg body weight, e.g. between 0.5 mg to 5 mg/kg body weight. If the regimen is a continuous infusion, doses should also be in the range of 1 μg to 10 mg per kg of body weight per minute. A preferred therapeutic dosage is one that achieves steady state blood levels between 15 μg/ml and 35 μg/ml for the biologically-active fusion proteins. Doses below or above the indicated exemplary ranges also are envisioned, considering the aforementioned factors.

The pharmaceutical composition contained additional biologically active agents, depending on the intended use of the pharmaceutical composition. These further biologically active agents are at least one of antibodies, antibody fragments, hormones, growth factors, enzymes, binding molecules, cytokines, chemokines, nucleic acid molecules, and drugs.

Certain embodiments of the invention herein provide methods of preventing and/or treating acute and chronic inflammation and autoimmune diseases by administering a prophylactic and/or therapeutic formulation containing recombinant sTNF-RI or sTNF-RII proteins which have been modified either by conjugating natural amino acids or a combination of natural and unnatural amino acids creating a linear polypeptide of specific length “n”. Further, the recombinant soluble human full-length sTNF-RI or sTNF-RII proteins were also modified by conjugating sugar molecules of heparosan to different regions of the protein. The use of naturally occurring sTNF-Rs allows for longer and more effective treatment since sTNF-Rs essentially cleave cellular receptors that are cleared normally in humans.

Certain embodiments of the invention herein use sTNF-RI or sTNF-RII as a targeting agent modified by natural amino acids or a combination of natural and unnatural amino acids of specific length “n” creating a linear polypeptide also incorporating anti-inflammatory drugs such as methotrexate to treat arthritis and other inflammatory diseases. A variety of steroidal drugs, non-steroidal drugs, and disease modifying drugs, and other anti-inflammatory compounds were also incorporated into the sTNF-Rs modified by conjugating natural amino acids or a combination of natural and unnatural amino acids of specific length “n” creating a linear polypeptide. The sTNF-Rs attached by conjugating natural amino acids or a combination of natural and unnatural amino acids of specific length “n” creating a linear polypeptide accumulate within the inflamed site where the drug is released for maximum therapeutic effect.

Use of sTNF-RI or sTNF-RII as a targeting agent attached to heparosan molecules also incorporating anti-inflammatory drugs such as methotrexate to treat arthritis and other inflammatory diseases is shown herein. A variety of steroidal drugs, non-steroidal drugs, disease modifying drugs, and other anti-inflammatory compounds are incorporated into the sTNF-Rs modified by heparosan molecules. The sTNF-Rs attached to heparosan molecules accumulate within the inflamed site where the drug is released for maximum therapeutic effect.

Certain embodiments of the invention herein use novel technologies to extend the half-lives of biopharmaceutical drugs so that they not only circulate longer in the body to treat the disease, but also do so in a stealthy manner so as not to be rejected by the body by an immune response.

In contrast to existing biopharmaceutical drugs for treating arthritis and other inflammatory diseases, certain embodiments of the invention herein masked the immuno-suppressive nature of a biopharmaceutical drug and simultaneously increased its half-life in the body. Consequently, it was not rejected by the body, did not result in immune reactions, and was dosed at lower quantities or frequency.

### Description of Process

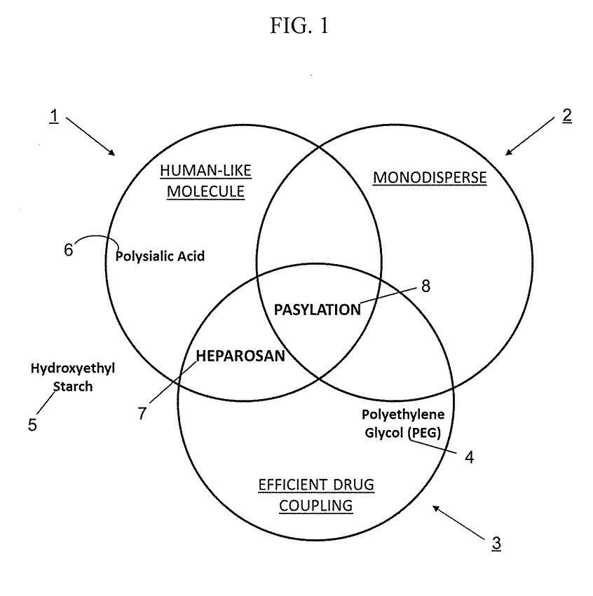

FIG. 1 schematically identifies problems of prior art methods and illustrates the desired operating characteristics and regime for an optimal solution. The solution is characterized as a human-like molecule, 1, capable of monodispersity, 2, and efficient drug coupling methods, 3, (either by means of genetic fusion or by chemical conjugation techniques). Chemical conjugation is performed, for example, by selective N-terminal chemical modification as described by Kinstler et al., U.S. Pat. No. 5,824,784 and U.S. Pat. No. 5,985,265. A water soluble polymer is attached to the N-terminus of the protein by performing the reaction at a pH which allows one to take advantage of the pKa differences between the ε-amino group of the lysine residues and that of the α-amino group of the N-terminal residue of the protein. Attachment of a water soluble polymer to a protein is controlled by selective derivatization. Conjugation with the polymer takes place predominantly at the N-terminus of the protein and no significant modification of other reactive groups occurs. Using reductive alkylation, the water soluble polymer has a single reactive aldehyde for coupling to the protein. A similar process is used for chemical conjugation to the C-terminus, or to residues which are internal to both the C-terminus and the N-terminus. Additional methods have been reviewed in Means et al., Bioconjugsyr Chem., 1, 2-12 (1990).

The PAS polypeptide or heparosan sugar chain used herein is made by any procedure available to one of skill in the art. For example, the PAS polypeptides or heparosan sugar chain is made under condensation conditions using the desired molar fraction of the component amino acids as precursors for polymerization, either in solution or by solid phase synthetic procedures. See, WO 2000/005250. All input desired molar ratios of the precursor monomer components to each other are envisioned herein as under control by the user.

For solution phase synthesis of the polypeptide polymer, condensation conditions include the proper temperature, pH, and solvent conditions for condensing the carboxyl group of one amino acid with the amino group of another amino acid to form a peptide bond. Condensing agents, for example, dicyclohexyl-carbodiimide, are used to facilitate the formation of the peptide bond. Blocking groups are used to protect functional groups, such as the side chain moieties and some of the amino or carboxyl groups, against undesired side reactions.

For example, N-carboxyanhydrides, γ-benzyls, and N-trifluoroacetyls of proline, alanine, and serine are polymerized at ambient temperatures in anhydrous dioxane with diethylamine as an initiator. See, U.S. Pat. No. 3,849,550 issued Nov. 19, 1974. The γ-carboxyl group is deblocked by hydrogen bromide in glacial acetic acid. The trifluoroacetyl groups are removed by one molar piperidine. One of ordinary skill in the art would understand that the process can be adjusted to make peptides and polypeptides containing the desired amino acids, for example, two of the three amino acid residues. For purposes of this application, the terms “ambient temperature” and “room temperature” mean a temperature ranging from about 20 to about 26 degrees ° C.

The average molecular weight of the resulting polypeptides polymer can be adjusted during or after synthesis. See, WO 2000/005250. To adjust the average molecular weight during polypeptide synthesis, the synthetic conditions or the amounts of amino acids are adjusted so that synthesis stops when the polypeptide reaches the approximate length which is desired. After synthesis, polypeptide polymers with the desired average molecular weight can be isolated from the reaction mixture by any available size selection procedure, for example, chromatography of the mixture on a molecular weight sizing column or gel, and collection of the average molecular weight ranges as desired. The resulting polypeptide polymer can also be partially hydrolyzed to remove high molecular weight species, for example, by acid or enzymatic hydrolysis, and then purified to remove the acid or enzymes.

Two primary forms of solid phase synthesis methods use Fmoc and Boc precursors. Small beads containing linkers on which peptide chains can be built. The N-termini of amino acid monomers is protected by Fmoc or Boc groups added onto a deprotected amino acid chain. The synthesis beads retain strong bondage to the peptides until cleaved by a reagent such as trifluoroacetic acid. The beads create a synthesis environment in which the peptide chains in the process of elongation are retained, viz., will not pass through a filter material, to separate these chains from the reagents used to synthesize them. Each amino acid is present in substantial excess (i.e. two to ten times) and coupling amino acids to form peptide bonds is highly optimized by a series of well-characterized agents. Unlike ribosome protein synthesis, solid-phase peptide synthesis proceeds in a C-terminal to N-terminal direction.

Solid phase synthesis is limited by yields accordingly is not used for synthesis beyond a particular length, e.g., typically peptides and proteins in the range of 70 to 100 amino acid residues are at the limit of synthetic accessibility. Longer lengths can be attained by using native chemical ligation to couple two peptides together with quantitative yields. Automated programmable synthesizers are available.

Prior art techniques for increasing the half-life of proteins include use of PEG, 4, hydroxyethyl starch, 5, and/or polysialic acid, 6. As noted in FIG. 1, each of these have characteristics that preclude them from providing the optimal half-life extension solution. While use of PEG, 4, is currently the most widespread half-life extension technology for biological molecules, the European Medicines Agency has released warnings associated with the long-term administration of drugs containing PEG, 4, because of increasing evidence of cellular vacuolation in various organs and in renal tubular cells²¹. Technologies using sugar molecules of heparosan, 7, and a technology called PASylation®, 8, have advanced the half-life extension/drug delivery frontier. Certain embodiments of the invention herein combined the characteristics of human-like molecules, 1, capable of monodispersity 2, and efficient drug coupling methods, 3, in an optimal manner, and as embodied by PASylation®, 8, used in modification of proteins such as sTNF-RI; sTNF-RII; and the 4 domain p55 sTNF-RI protein, to circumvent performance issues of the prior art methods.

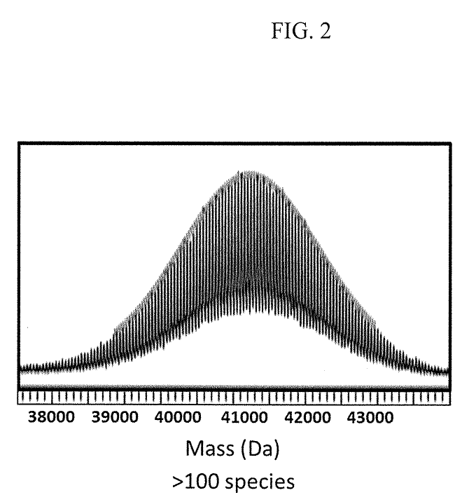

FIG. 2 is a graph of de-convoluted zero-charge mass spectra showing the highly polydisperse nature of the currently available technology using PEG residues that increased the half-life of biopharmaceutical drugs. See, Bagal et al., Anal. Chem., 80: 2408-2418 (2008). On account of the highly polydisperse nature of PEG, when conjugated to a drug, the PEG masks the reactive site of the drug, which results in a dramatic reduction in the effectiveness of the drug.

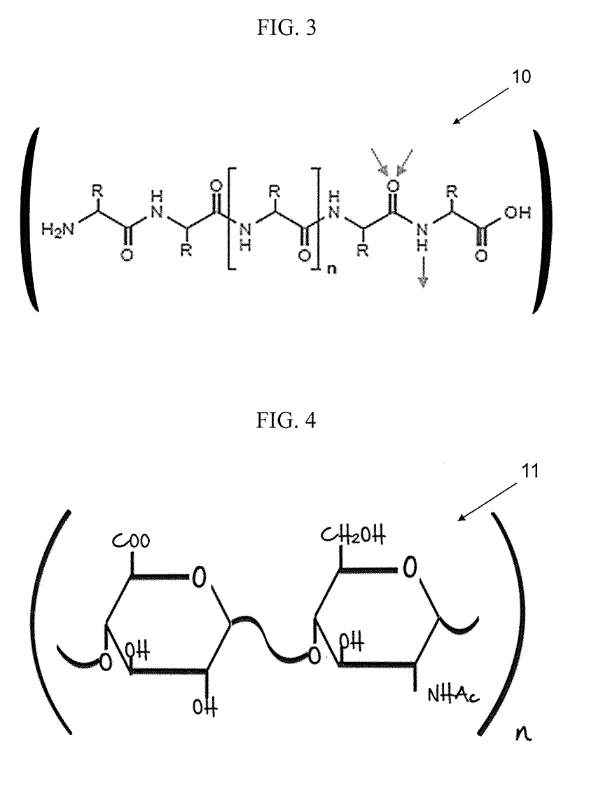

FIG. 3 is the basis of the PASylation® technology and depicts the structure and sequence of natural amino acids, containing a PA/S creating a polypeptide, 10, of specific length “n”, such that “n” varies. The lengths of the polypeptide varied from 100 amino acid residues up to 1,200 amino acid residues or more. The actual length chosen depends on the half-life extension being desired, and the number of amino acid residues is potentially greater than 1200. Because the PAS polypeptide, 10, contained natural amino acids, the body did not recognize it as foreign and hence does not elicit an immune-response signal, unlike the results of administration of PEG, 4. The polypeptide may be combined with unnatural amino acids, if a particular function is desired. An advantage of the PAS polypeptide, 10, is that it can be genetically fused to the biopharmaceutical drug for simultaneous expression, or it can be chemically conjugated, unlike the other technologies in FIG. 1.

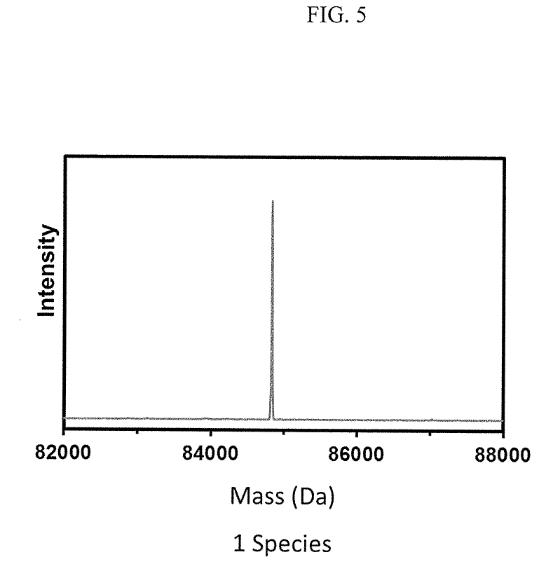

FIG. 4 depicts a molecular unit of a structure formed by repeating units of sugar molecules of heparosan, 11. Unlike the PAS polypeptide, 10, heparosan molecules were chemically conjugated to the drug, and cannot be expressed by genetic means. Heparosan molecules were attractive for the purposes of half-life extension and drug delivery modification for a number of reasons. Heparosan is a substance already present in the human body. Certain bacteria coat themselves with heparosan so that they are camouflaged from the immune-response system of the human body. Hence, heparosan molecules, 11, have the potential to act as a stealth molecule for drug delivery purposes. Heparosan molecules have less homogeneity than the PAS polypeptide, 10, but because multiple units of the sugar molecules were chemically conjugated, it provide better control of polydispersity than PEG, 4.

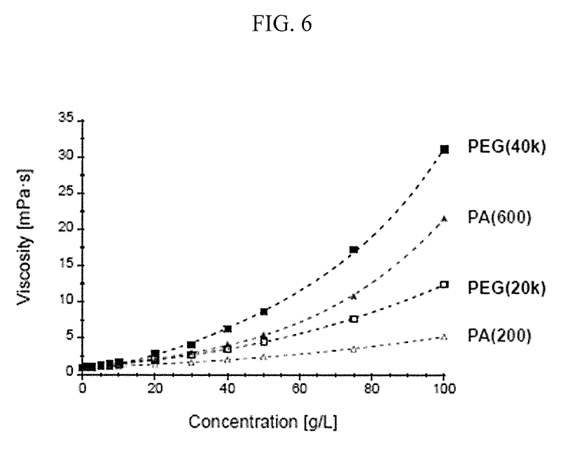

FIG. 5 is a graph of mass spectroscopy data showing the single-species level of homogeneity and monodisperse nature of the PAS polypeptide, 10.

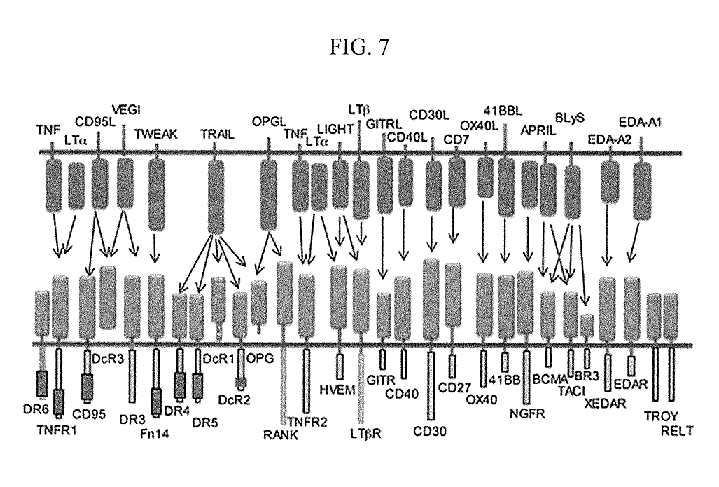

FIG. 6 compares the viscosities among various lengths of polypeptides containing amino acid residues, which are exemplified by the repeat structure of FIG. 3 and PEG polymers in the preferred molecular weight range. The viscosities were measured with a microviscometer with VROC® chip in phosphate buffered saline. Neither the PA/S polypeptide chain nor the PEG polymers were fused or conjugated to proteins as partners, and FIG. 6 depicts the inherent baseline viscosities. Viscosities of PASylated or PEGylated drugs are influenced by fusion and conjugation partner(s). The hydrodynamic volumes of the PA(200) polypeptide chain roughly corresponds to a PEG polymer of molecular weight 20 kDa, while that of the PA(600) polypeptide chain roughly corresponds to a PEG polymer of molecular weight 40 kDa. The data provide that for corresponding hydrodynamic volumes at the higher concentrations, the PAS polypeptides have viscosities that are one-third to three-fold lower than the PEG polymers.

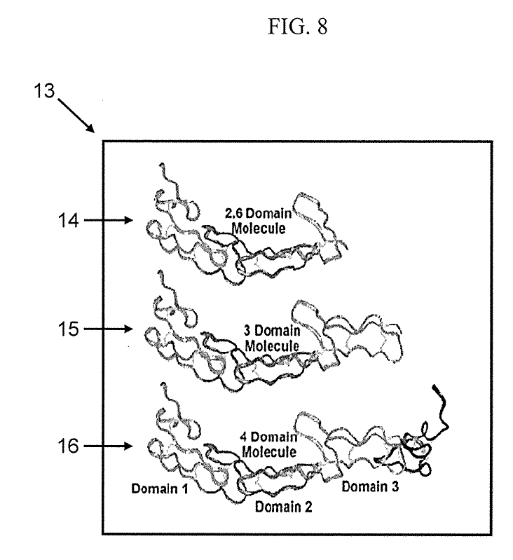

FIG. 7 is a diagram of members of the TNF receptor superfamily and known ligands of each. Many ligands have been observed to bind to more than one receptor as indicated by arrows. Ligands for DR6, TROY and RELT have not yet been discovered. Dark boxes shown in the cytoplasmic part of the receptors indicate presence of death receptor domains. Death receptor refers to members of the TNF receptor superfamily that contain a death domain, exemplified by family members such as TNF-RI, Fas receptor, DR4, and DR5. These receptors have been observed to function in apoptosis (programmed cell death), in addition to other roles.³⁸

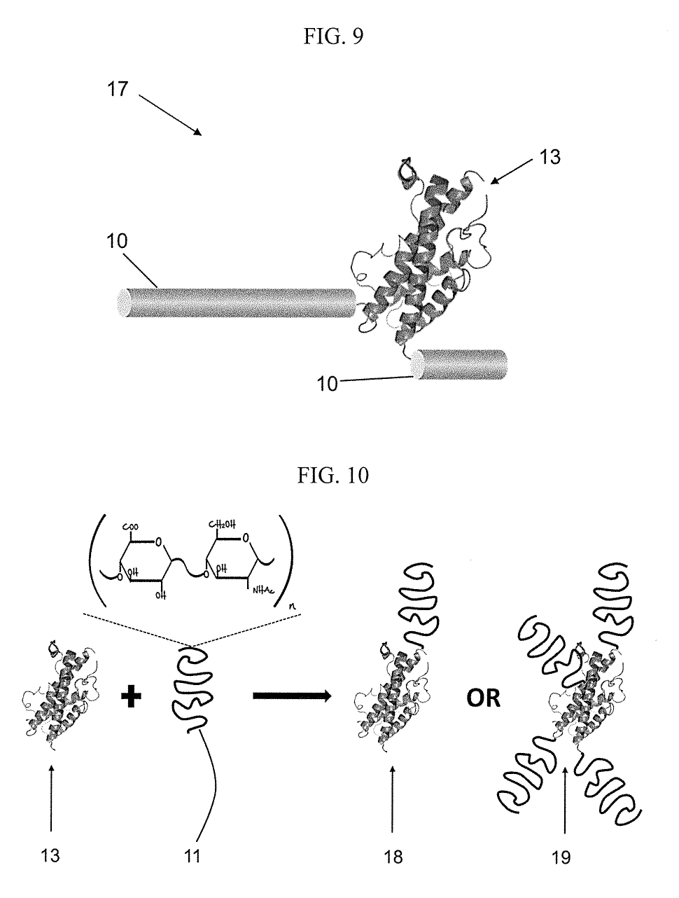

FIG. 8 are x-ray crystallography data providing the three-dimensional structures of biologically-active forms of sTNF-Rs, 13, containing the following protein domains and variants of the human sTNF-Rs—2.6 domains, 14, 3.0 domains, 15, and 4.0 domains, 16. The protein containing 4.0 domains, 16, is the human wild-type sTNF-R.

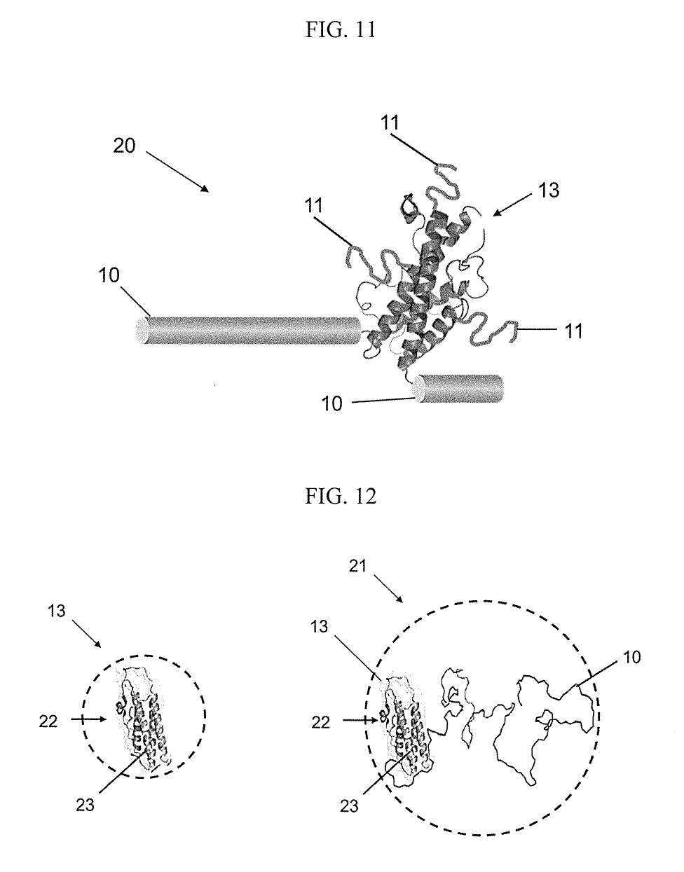

FIG. 9 is a drawing of the structure of a pharmaceutical composition, 17, containing a biologically-active form of sTNF-Rs, 13, conjugated to PAS polypeptides, 10. The biologically-active form of the sTNF-Rs, 13, was combined with the PAS polypeptides, 10, or variants thereof by classical molecular biology techniques or by classical chemical reactions. Conjugation is feasible for either the N-terminus of the biologically-active forms of sTNF-Rs, 13, and/or the C-terminus of the biologically-active forms of sTNF-Rs, 13.

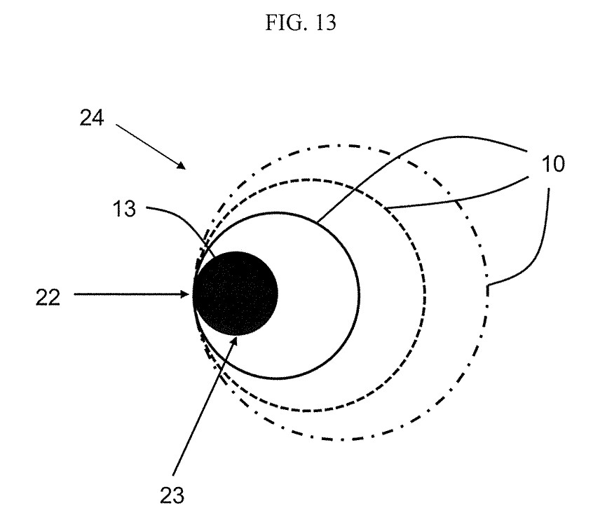

FIG. 10 is a drawing in schematic view the creation of new biological entities: a biologically active form of sTNF-Rs, 13, conjugated to heparosan molecules at one position on the protein to form a pharmaceutical composition, 18, and a biologically active form of the sTNF-Rs, 13, conjugated to heparosan molecules at more than one position on the protein to form a pharmaceutical composition, 19. Conjugation of a least one biologically-active form of the sTNF-Rs, 13, with heparosan molecules, 11, and its variants was performed by chemical conjugation techniques known in the art.

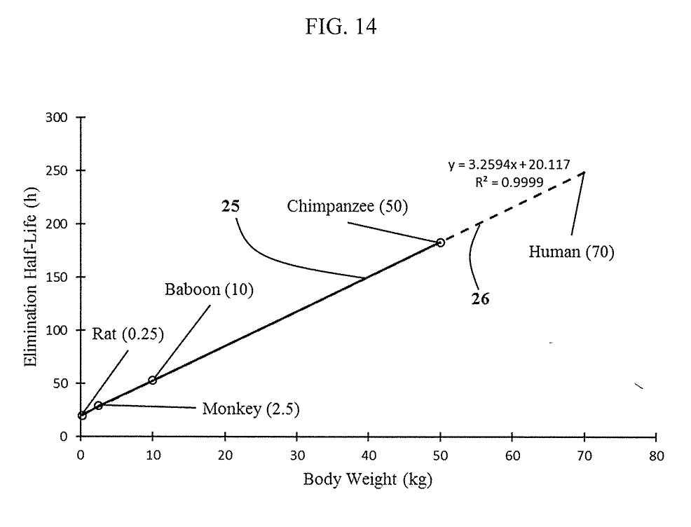

FIG. 11 shows a further embodiment of the invention in which a least one biologically-active form of the sTNF-Rs, 13, is combined with a PAS polypeptide, 10, and its variants and heparosan molecules, 11, and its variants.

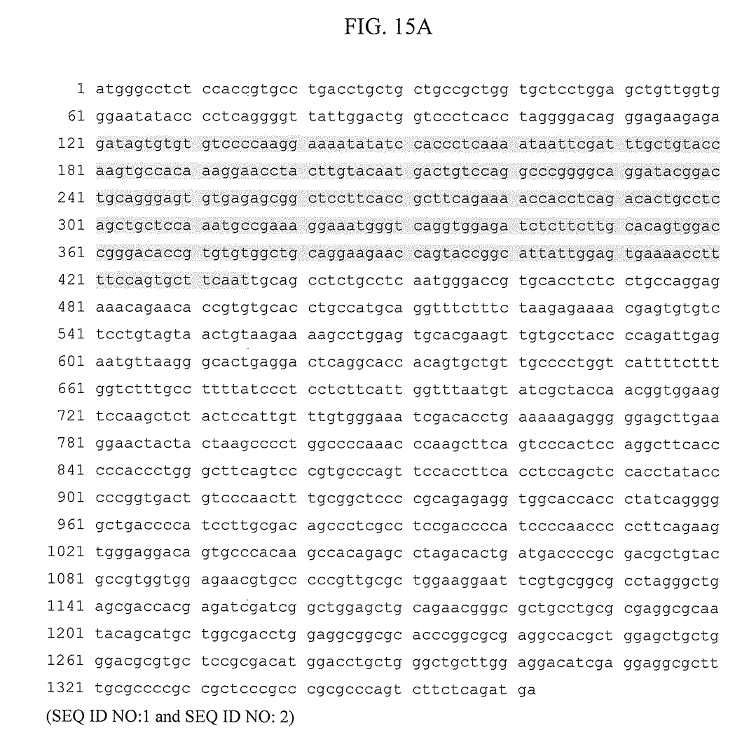

FIG. 12 is a drawing of a composite view, 21, of the net effective increase in the molecular or hydrodynamic volume of the biologically-active forms of sTNF-Rs, 13, when conjugated by either genetic or chemical methods to a PAS polypeptide, 10. Such conjugation achieves two desired goals simultaneously—the reactive site, 22, in the biologically-active forms of sTNF-Rs, 13, remains open and unhindered, and the immunogenic sites on the biologically-active forms of sTNF-Rs, 13, are masked by the picosecond to femtosecond vibrations of the PAS polypeptide, 10, and/or its variants. These characteristics provide clinical benefits to patients.

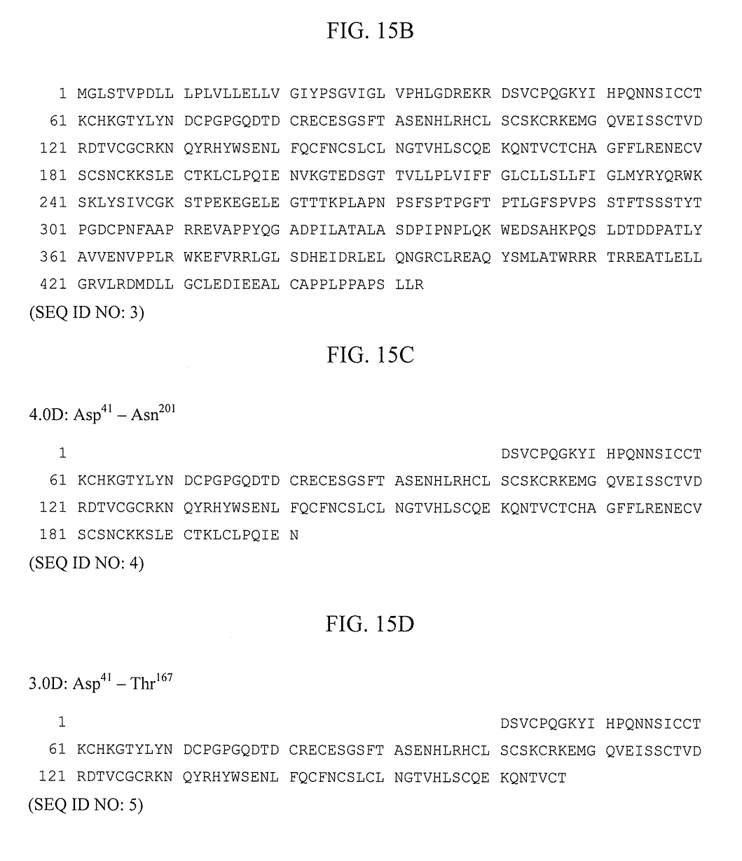

FIG. 13 is a drawing of the beneficial effects of FIG. 11, and contains the net effect on the hydrodynamic volume of the biologically-active forms of sTNF-Rs, 13, by increasing the number of amino acid residues in the PAS polypeptide 10, with the increase in circle diameters correlating to increasing lengths of the PAS polypeptide.

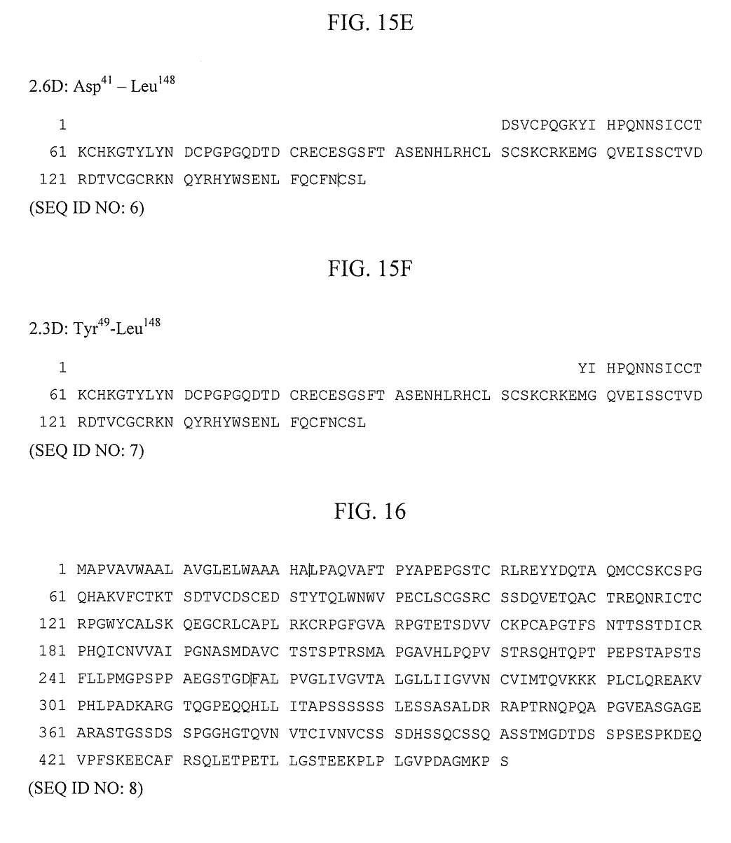

FIG. 14 is a graph of the elimination half-life of pharmaceutical composition, 17, versus body weight. The volume of distribution and plasma clearance of protein pharmaceuticals over a wide molecular weight range (6,000 to 98,000 Daltons) followed size-related physiological relations. Preclinical pharmacokinetic studies provided reasonable estimates of human disposition after interspecies scaling³⁶. The elimination half-life/plasma clearance data for the pharmaceutical composition, 17, were scaled from rats, monkeys, baboons, and chimpanzees, 25, to predict the pharmacokinetics in humans, 26. However, as chimpanzees (Pan troglodytes) are the closest relative to humans of the animals and are of a similar body weight (50 kg), the pharmacokinetics in chimpanzees are expected to be similar to those in humans. Therefore, for a 70 kg human, the elimination half-life of a pharmaceutical composition containing a biologically active form of the sTNF-Rs conjugated to heparosan molecules, 20, was predicted to be about 250 hours. The correlation coefficient between actual data, 25, and the prediction for the half-life of the pharmaceutical composition, 17, in humans, 26, is calculated using the equation shown in FIG. 13.

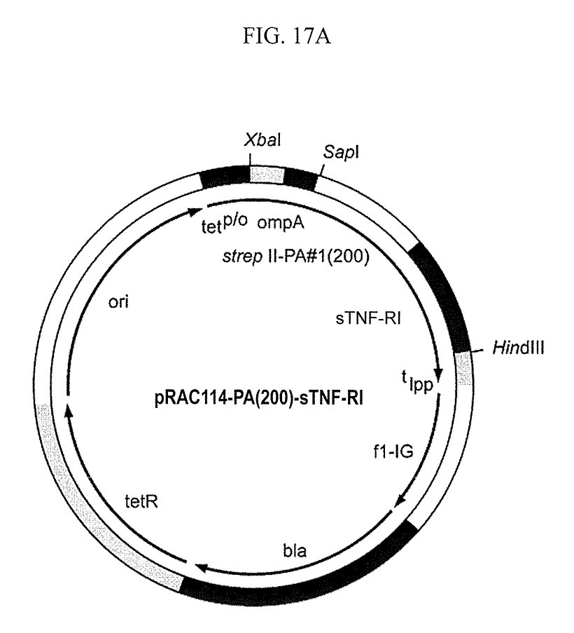

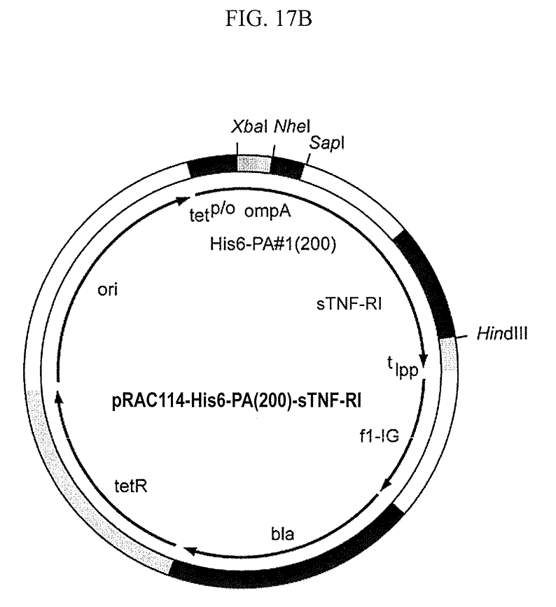

FIG. 15A is the nucleic acid sequence (SEQ ID NO: 1) of the full-length form of the sTNF-RI protein. The recombinant human sTNF-RI protein consists of 1,362 base-pairs (bp)³⁷. FIG. 15B is the amino acid sequence (SEQ ID NO: 3), GenBank Accession No.: AAA36756.1, translation of the nucleic acid sequence of FIG. 15A³⁷. FIG. 15E is the amino acid sequence (SEQ ID NO: 6) of the 2.6D protein (aa⁴¹-aa¹⁴⁸) created from the sTNF-RI full-length protein employing the molecular biology techniques described in detail herein above. This amino acid sequence is exemplary, and is not limiting to the particular domain that is extracted from the sTNF-Rs. For example, the 4.0D sTNF-RI protein contains the domains, for example but not limited to, domains 3.0D, 2.0D, and 1.0D. Each major domain unit contains sub-domains such as but not limited to 2.9D, 2.8D, 2.1D, and 2.0D. FIG. 15E is an amino acid sequence containing 2.6 domains (2.6D) (SEQ ID NO: 4) of the full-length sTNF-RI protein (SEQ ID NO: 3). FIG. 15C is an amino acid sequence from amino acid position 41 to amino acid position 201 of the full-length sTNF-RI protein (SEQ ID NO: 3) containing 4.0 domains (4.0D) (SEQ ID NO: 4). FIG. 15D is an amino acid sequence from amino acid position 41 to amino acid position 167 of the full-length sTNF-RI protein (SEQ ID NO: 3) containing 3.0 domains (3.0D) (SEQ ID NO: 5). FIG. 15F is an amino acid sequence from amino acid position 49 to amino acid position 148 of the full-length sTNF-RI protein (SEQ ID NO: 4) containing 2.3 domains (2.3D) (SEQ ID NO: 7). FIG. 16 is an amino acid sequence of the full-length sTNF-RII protein, NCBI Accession No.: NP_001057 (SEQ ID NO: 8).

Each of these domains demonstrates varying levels of biological activity by ability to inhibit the activity of TNFα and thereby provide a therapeutic benefit to the patient. Each of these individual domains ranging from 1.0D through 4.0D are suitably modified either at its N-terminus, or at its C-terminus, or at both termini, using the technique of PASylation® to obtain a tunable half-life by design.

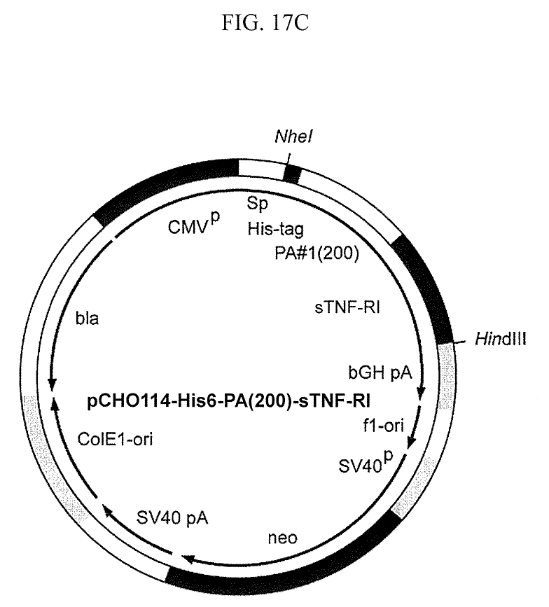

FIG. 17A is a schematic illustration of a typical clone construct and plasmid map of a PAS polypeptide with 200 amino acid residues fused to a sTNF-RI protein (pRAC114-PAS200-sTNF-RI) for simultaneous expression in a prokaryotic system such as E. coli. PA/S gene cassettes expressing PA/S polypeptides of various lengths ranging from at least 100 amino acid residues to well over 1,200 amino acid residues. These cassettes are commercially available from XL-protein GmbH, Lise-Meitner-Straβe 30, 85354 Freising, Germany. Full-length or truncated biologically-active forms of sTNF-Rs, 13, were prepared as detailed herein. The structural gene for pRAC114-PAS200-sTNF-RI contains the following functional groups: the bacterial OmpA signal peptide, the Strep-tag II, the PA/S polymer with 200 residues (PAS(#1)200), and human sTNF-RI. The entire amino acid sequence is under transcriptional control of the tetracycline promoter/operator)(tet^{p/o}) and terminates with the lipoprotein terminator (tᵢₚₚ). The plasmid backbone, i.e. outside the expression cassette flanked by the XbaI and HindIII restriction sites, is a generic cloning and expression vector³⁸. Singular restriction sites are indicated in FIG. 17A and FIG. 17B. The expression vectors for PAS400-, PAS600-, PAS800-, PAS1,000-, or PAS1,200-sTNF-RI are identical except that these contain, respectively, the PAS#1 polymer with 400-, 600-, 800-, 1,000- or 1,200 amino acid residues or more, is encoded by a corresponding gene cassette instead of PAS(#1)200. An exemplary amino acid sequence of PAS#1 is ASPAAPAPASPAAPAPSAPA (SEQ ID NO: 9).

In additional embodiments, the sequence contains conservative amino acid mutations, which are mutations that change an amino acid to a different amino acid with similar biochemical properties, for example, the properties of charge, hydrophobicity, and size. For example, leucine and isoleucine are both aliphatic, branched, and hydrophobic. Similarly, aspartic acid and glutamic acid are both small, negatively charged residues. Conservative mutations in proteins often have a smaller effect on function than non-conservative mutations.

Amino acids are classified into six main groups on the basis of their structure and the general chemical characteristics of their R groups:

Aliphatic—glycine (G), alanine (A), valine (V), leucine (L), isoleucine (I) Hydroxyl or sulfur-containing—serine (S), cysteine (C), threonine (T), methionine (M)

Cyclic—proline (P)

Aromatic—phenylalanine (F), tyrosine (Y), tryptophan (W)

Basic—histidine (H), lysine (K), arginine (R)

Acidic and Amide—aspartate (D), glutamate (E), asparagine (N), glutamine (Q)

FIG. 17B is an alternative embodiment of the plasmid map of a PAS polypeptide with 200 amino acid residues fused to a sTNF-RI molecule (pRAC114-His6-PA200-sTNF-RI) for simultaneous expression in prokaryotic systems such as E. coli. The PA/S cassette, which is commercially available from XL-protein GmbH, Lise-Meitner-Straβe 30, 85354 Freising, Germany, has an affinity tag containing a histidine polypeptide with at least six residues (e.g. His₆-PA#1(200)), which functions to specifically aid in the subsequent chromatographic purification of the sTNF-RI using well-established metal-chelate affinity chromatographic techniques.⁴⁴ Additional embodiments can include several other tags known in the art, as part of the gene fusion to simplify the purification process.

FIG. 17C is an embodiment of a plasmid map (pCHO114-PA(200)-sTNF-RI) for the secretory production of a fusion product of a sTNF-R and a genetically encoded PA/S polypeptide with 200 amino acid residues for simultaneous expression in eukaryotic systems such as CHO cells. The plasmid map of pCHO114-PA(200)-sTNF-RI encodes a His₆-PA#1(200)-sTNF-RI fusion protein. The His₆-PA#1(200) cassette, which is commercially available from XL-protein GmbH, Lise-Meitner-StraBe 30, 85354 Freising, Germany, has an affinity tag containing a histidine polypeptide with at least six residues, which aids in the subsequent chromatographic purification of the sTNF-RI using metal-chelate affinity chromatographic techniques.⁴⁴ The structural gene contains the sTNF-RI signal peptide (Sp), the His₆-tag, the PA#1 polymer/polypeptide sequence with 200 residues (PA#1(200)), the sTNF-RI, and the bovine growth hormone polyadenylation signal (bgh-PolyA) to achieve a high level of expression of peptides in eukaryotic cells, is under transcriptional control of the cytomegalovirus promoter (CMVP). See, U.S. Pat. No. 5,122,458. The singular restriction sites NheI and HindIII are indicated. The resistance gene for neomycinphosphotransferase (neo) is under control of the SV40 promotor (SV40^{p}) and followed by a SV40 polyadenylation signal (SV40 pA). Additionally, the plasmid contains the bacterial ColE1 origin of replication (ColE1-ori), the bacteriophage fl origin of replication (fl-ori), and the β-lactamase gene (bla) to allow propagation and selection of the plasmid in E. coli.

By following the steps described above and from knowledge of basic molecular biology techniques (Sambrook et al.)³⁰ and chemical reactions (for example, thiol-, or alkyl-, or aldehyde chemistries), one of ordinary skill in the art could make and use an embodiment of the invention as described herein.

Recombinant human sTNF-RI 4.0D, 16, is commercially available (e.g. SRP4348-sTNF-RI human, Sigma-Aldrich). The source of 4.0D, 16, was prokaryotic-, eukaryotic- or plant-based host vehicle capable of expressing the protein with fidelity. The expression hosts are for example, but not limited to bacterial cells such as E. coli, mammalian cells such as Chinese Hamster Ovary (CHO) cells, yeasts, baculovirus, tobacco mosaic virus, and plant cells. PAS polypeptides of various lengths of at least 100 amino acid residues is commercially available from XL-protein GmbH, Lise-Meitner-StraBe 30, 85354 Freising, Germany, and heparosan molecules, 11, having a molecular weight in the range of about 20,000 Daltons to about 60,000 Daltons is commercially available from Caisson Biotech, Austin, Tex. Alternatively, the heparosan molecules have a molecular weight of over 60,000 Daltons. The PAS polypeptide 10 was combined with biologically-active forms of sTNF-Rs, 13, by genetic fusion based on molecular biology techniques or by chemical conjugation, and heparosan molecules, 11, were conjugated to biologically-active forms of sTNF-Rs, 13, by chemical conjugation.

Expression of a PASylated form of one of the domain-forms of sTNF-Rs is a process familiar to one of ordinary skill in the art. The genetic fusion of a PAS sequence with any one of the forms of sTNF-Rs was expressed either in the cytoplasmic space of an E. coli host, or in the periplasmic space of E. coli. Alternatively, other expression hosts (e.g. CHO) were also considered. For periplasmic expression, a nucleic acid sequence such ‘ATG’ was added as a start codon to the N-terminus of the sTNF-R gene of interest. The start codon was followed by a signal peptide such as the OmpA periplasmic signal sequence, which was followed by two unique type IIS SapI restriction sites upstream of the sTNF-R gene sequence. A stop codon for example but not limited to the nucleic acid sequence ‘TAA’ was added at the C-terminus of the sTNF-R gene. Using a combination of restriction enzymes and ligases, the SapI sequence was spliced out, leaving the classical “sticky” ends behind. The PAS gene sequence cassette with complimentary “sticky” ends was inserted by ligation to create the PAS-sTNF-R gene to be inserted by known plasmid-insertion techniques into the appropriate host for expression of a PAS-modified sTNF-R protein.

Biologically-active forms of sTNF-Rs, 13, PAS polypeptide, 10, and/or heparosan molecules, 11, as used in the relationship combinations described herein improved function of an embodiment of the invention herein. A variety of steroidal drugs, non-steroidal drugs, disease modifying drugs, and other anti-inflammatory compounds are incorporated into the sTNF-Rs modified by conjugating either natural amino acids or a combination of natural and unnatural amino acids creating a polypeptide chain of specific length “n”, or by the heparosan sugar molecules.

### How to Use Embodiments of the Invention

Certain embodiments of the invention described herein would be used by medical doctors and practitioners to treat patients suffering from life-long diseases or conditions of inflammation and immunology such as diabetic retinopathy and arthritis.

### Examples (1-6)

TABLE 1

| | | Half-Life of | |
|---|---|---|---|
| | | Modified | Fold |
| Ex. | | Molecule | Increase |
| No. | Protein Sample | (h) | in Half-Life |
| 1 | Unmodified antibody fragment (Fab) | 1.3 | 1.0 |
| 2 | Fab with 1 backbone of 100 PAS residues | 2.7 | 2.0 |
| 3 | Fab with 1 backbone of 200 PAS residues | 5.2 | 3.9 |
| 4 | Fab with 1 backbone of 400 PAS residues | 14.4 | 10.7 |
| 5 | Fab with 1 backbone of 600 PAS residues | 28.2 | 21.0 |
| 6 | Fab with 2 backbones of 200 PAS residues each | 37.2 | 27.8 |

### Effect of Increasing the PASylation® Residues on the Half-Life of the Payload

The data in Table 1 provides the effect of increasing the number of PAS residues on the half-life of a model antibody fragment (Fab). As shown in Examples 1-5, there was a correlation between increasing the number of PAS residues and increasing the half-life of the Fab. Example 6 showed a slightly different trend, two polypeptides of 200 PAS residues resulted in a greater increase in the half-life of the Fab, than one polypeptide of 400 residues. The two polypeptides of 200 residues were each conjugated to two different locations on the Fab, which created a larger effective molecular volume than just one polypeptide of 400 PAS residues. Antibody-type proteins provide for multiple locations for conjugation. The range of PAS residues shown in Table 1 are exemplary and are not restrictive, as amino acid residues may be added to extend the polypeptide well beyond 1,200 amino acid residues. The length of the PAS polypeptide is restricted by the particular clinical results required of each payload.

### Examples (7-14)

TABLE 2

| | | | Half- Life of | Half-Life of | |
|---|---|---|---|---|---|
| | | | Unmodified | Modified | Fold |
| Ex. | | | Molecule | Molecule | Increase |
| No. | Protein Sample | Study Model | (h) | (h) | in Half-Life |
| 7 | OmC1 + 600 PAS polypeptide | Mouse | 0.28 | 4.29 | 15 |
| 8 | Leptin + 600 PAS polypeptide | Mouse | 0.43 | 19.6 | 46 |
| 9 | IFNa2b + 600 PAS polypeptide | Mouse | 0.52 | 26.0 | 50 |
| 10 | IFNanta + 600 PAS polypeptide | Monkey | 0.28 | 19.4 | 69 |
| 11 | hGH + 600 PAS polypeptide | Mouse | 0.05 | 4.42 | 88 |
| 12 | Exendin + 600 PAS polypeptide | Mouse | 0.17 | 16.1 | 95 |
| 13 | sTNFRI + 30 kDa PEG polymer | Human | 0.85 | 82 | 96 |
| 14 | sTNFRI + 600 PAS polypeptide | Human* | 0.85 | >216 | >254 |

*On the basis of interspecies allometric scaling as shown in FIG. 12 for a 60 kg body weight.

### Impact of PASylation® on Different Payloads

Table 1 assessed the effect of varying lengths of PAS polypeptides on the half-life of a common payload. Examples in Table 2 documented the effect of using one type and length of PAS residues (PAS 600) on different payloads. The data were ranked in terms of Fold Increase in Half-Life (far right column). The nature and type of payload under consideration for half-life modification influenced the resulting half-life of the modified payload. The half-life of unmodified, non-antibody type proteins were observed to be in a relatively narrow range of typically less than one hour.

Examples 13 and 14 in Table 2 provides the half-life modification of 2.6D, 14, by two different technologies—Example 13 by PEGylation and Example 14 by PAGylation®. Example 13 (sTNFRI+30 kDa PEG polymer) is PEGsunercept®, the development of which appears to have been terminated or suspended in spite of having achieved positive human clinical phase II data. The viscosity of the combined 30 kDa PEG with the 2.6D, 14, protein made the pharmaceutical composition nearly glue-like in consistency (ca. 400 cP), thereby not only rendering its preparation and formulation for injection an extremely difficult task to accomplish, but also having a high associated cost of goods factor. As presented heretofore, the PAS polypeptide does not have the same viscosity issues as PEGylation. Furthermore, PASylation® has a lower associated cost of goods than PEGylation because the preferred mode for expression of the PAS polypeptide is simultaneously with the protein as a fusion product.

Based on the principles of interspecies allometric scaling illustrated in FIG. 13, the pharmaceutical composition in Example 14 was predicted to have a half-life in humans of at least 216 hours. This is a substantial advance and is of major importance and relevance in the improvement of treatments for arthritis and related autoimmune diseases with concomitant improvements in patient compliance, cost of treatment, and clinical burden. A current, established treatment for RA is etanercept (Enbrel®; Amgen, Inc., Thousand Oaks, Calif.). Enbrel® is a fusion protein of one variant of a biologically-active form of sTNF-Rs, 13, which is not PEGylated, and has a half-life of about 72 hours in humans³⁹, which is 10 hours less than that of the pharmaceutical composition Example 13.

Example 14 provides data that alteration of the dynamics of treatment and compliance for patients suffering from RA and chronic inflammation-related diseases. A half-life of around 216 hours in humans renders reduced dosing frequency of only once per two weeks, thereby providing the potential of a long-term (greater than three to five years) benefit to patients. Dose-creep with existing treatments develops after about six months of treatment. Benefits that would accrue as a consequence of this invention are an increase in patient compliance; considerably reduced clinical burden; and reduced cost of treatment, each of which would decrease the burden of increasing of healthcare costs.

### REFERENCE LIST

1. Joussen A M, Poulaki V, Mitsiades N, Kirchhof B, Koizumi K, Dohmen S, Adamis A P. Nonsteroidal anti-inflammatory drugs prevent early diabetic retinopathy via TNF-alpha suppression. FASEB J. 16:438-440. 2002.
2. Limb G A, Chignell A H, Green W, LeRoy F, Dumonde D C. Distribution of TNF alpha and its reactive vascular adhesion molecules in fibrovascular membranes of proliferative diabetic retinopathy. Br J Ophthalmol. 80:168-173. 1996.
3. Demircan N, Safran B G, Soylu M, Ozcan A A, Sizmaz S. Determination of vitreous interleukin-1 (IL-1) and tumour necrosis factor (TNF) levels in proliferative diabetic retinopathy, Eye. 20:1366-1369. 2006.
4. Cunha-Vaz J, Bernardes R. Nonproliferative retinopathy in diabetes type 2. Initial stages and characterization of phenotypes, Prog Retin Eye Res. 24:355-377. 2005.
5. Sedger L M & McDermott M F. TNF and TNF-receptors: From mediators of cell death and inflammation to therapeutic giants—past, present and future. Cytokine and Growth Factor Reviews 25, 453-472. 2014.
6. Schottelius A. et al. Biology of tumor necrosis factor-α (TNF-α): Implications for psoriasis. Experimental Dermatology 13, 193-222. 2004.
7. Christen U, Thuerkauf R, Stevens R & Lesslauer W. Immune response to a recombinant human TNFR55-IgG1 fusion protein: Auto-antibodies in rheumatoid arthritis (RA) and multiple sclerosis (MS) patients have neither neutralizing nor agonist activities. Human Immunology 60, 774-790. 1999.
8. Furst D, Weisman M, Paulus H & et al. Neutralization of TNF by Lenercept (TNFᵣ₅₅₋IgG₁, Ro 45-2081) in patients with rheumatoid arthritis treated for 3 months; results of an US phase II trial. Arthritis and Rheumatism 39, 5243. 1996.
9. Sander 0 et al. Neutralization of TNF by Lanercept (TNF_{R55-}IgG₁, Ro 45-2081) in patients with rheumatoid arthritis treated for 3 months: results of a european phase II trial. Arthritis and Rheumatism 39, 5242. 1996.
10. Solorzano C C et al. Involvement of 26-kDa cell associated TNFα in experimental hepatitis and exacerbation of liver injury with a matrix metalloproteinase inhibitor. J-Immunol. 158, 414-419. 1997.
11. Moreland L W et al. Phase I/II trial of recombinant methionyl human tumor necrosis factor binding protein PEGylated dimer in patients with active refractory rheumatoid arthritis. J-Rheumatol. 27, 601-609. 2000.
12. McCabe D et al. A phase I/II study to evaluate the safety, immunogenicity, pharmacokinetics and potential efficacy of IV rHuTNF binding protein pegylated dimer (TNFbp) in patients with active R A. Arthritis and Rheumatism 41, S58. 1998.
13. Edwards III C K. PEGylated recombinant human soluble tumour necrosis factor receptor type I (r-Hu-sTNF-RI) novel high affinity TNF receptor designed for chronic inflammatory diseases. Annals of Rheumatic Diseases 58, 173-181. 1999.
14. Jones M D et al. Determination of tumor necrosis factor binding protein disulfide structure: deviation of the fourth domain structure form tnfr/ngrf family cysteine-rich region signature. Biochemistry. 36, 14914-14923. 1997.
15. Furst D et al. A phase 2 dose-finding study of PEGylated recombinant methionyl human soluble tumor necrosis factor type I in patients with rheumatoid arthritis. The Journal of Rheumatology. 1-31. 2005.
16. Glatt S, Fuseau E, Buraglio M & Nguyen QTX. Population pharmacokinetics of onercept in healthy subjects. Clin Pharmacokinetics 44[12], 1295-1304. 2005.
17. Rutgeerts P et al. Treatment of active Crohn's disease with onercept (recombinant human soluble p55 tumour necrosis factor receptor): results of a randomized, open label, pilot study. Ailment Pharmacol Ther. 17, 185-192. 2003.
18. Engelmann H, Aderka D, Rubinstein M, Rotman D & Wallach D. A tumor necrosis factor binding protein purified to homogeneity human urine protects cells from tumor necrosis factor toxicity. J-Biol-Chem. 264, 44974-11980. 1989.
19. Novick D et al. Purification of soluble cytokine receptors from normal human urine by ligand-affinity and immunoaffinity chromatography. Journal of Chromatography B: Biomedical Applications 510, 331-337. 1990.
20. Novick D, Engelmann H, Wallach D & Rubinstein M. Soluble cytokine receptors are present in normal human urine. J-Exp-Med. 170, 1409-1414. 1989.
21. European Medicines Agency. CHMP Safety Working Party's rsponse to the PDCO regarding the use of PEGylated drug products in the pediatric population. EMA/CHMP/SWP/647258/2012. 16 Nov. 2012.
22. Smith et al. Science. 248:1019-1023. 1990.
23. Corcoran et al. Eur. J. Biochem. 223:831-840. 1994.
24. Chih-Hsueh et al. The Journal of Biological Chemistry. 270(6):2874-2878. 1995.
25. Scallon et al. Cytokine, 7(8):759-770. 1995.
26. Schlapschy M, Binder U, Borger C, Theobald I, Wachinger K, Kisling S, Haller D & Skerra A. PASylation: a biological alternative to PEGylation for extending the plasma half-life of pharmaceutically active proteins. Protein Engineering, Design & Selection 26:8 489-501. 2013.
27. Morath V, Bolze F, Schlapschy M, Schneider S, Sedlmayer F, Seyfarth K, Klingenspor M, & Skerra A. PASylation of Murine Leptin Leads to Extended Plasma Half-Life and Enhanced in Vivo Efficacy. pubs.acs.org/molecularpharmaceutics. 2015.
28. Skerra et al., WO 2008/155134Δ1. Biological active proteins having increased in vivo and/or vitro stability. Published Dec. 24, 2008.
29. Skerra et al., WO 2011/144756Δ1. Biosynthetic proline/alanine random coil polypeptides and their uses. Published Nov. 24, 2011.
30. Sambrook et al. Molecular Cloning: A Laboratory Manual. Cold Spring Harbor Laboratory Press, Cold Spring Harbor, N.Y. 1989.
31. Ausubel et al., eds. Current Protocols in Molecular Biology. Current Protocols Press. 1994.
32. Berger and Kimmel. Methods in Enzymology: Guide to Molecular Cloning Techniques. Vol. 152, Academic Press, Inc., San Diego, Calif. 1987.
33. Engels et al. Angew. Chem. Intl. Ed. 28:716-734. 1989.
34. Wells et al. Gene. 34:315. 1985.
35. Remington's Pharmaceutical Sciences. 22nd edition, Osol, A. Ed. 2012.
36. Grene-Lerouge et al. Interspecies scaling of clearance and volume of distribution for digoxin-specific Fab. Toxicol. Appl. Pharmacol. 138, 84. 1996.
37. Gray, P. W., Barrett, K., Chantry, D., Turner, M. and Feldmann, M. Cloning of human tumor necrosis factor (TNF) receptor cDNA and expression of recombinant soluble TNF-binding protein. Proc. Natl. Acad. Sci. U.S.A. 87 (19), 7380-7384. 1990.
38. Skerra A. Gene 151:131-135. 1994.
39. https://en.wikipedia.org/wiki/Etanercept. 2015.
40. Palladino, M. A., et al. Nat. Rev. Drug Discov. 2:736-46. 2003.

What is claimed is:

1. A composition for preventing or treating a subject for at least one of an inflammation, an autoimmune disease, and a metabolic disease, the composition comprising:
a full-length or a truncated form of a receptor protein that is a member of the superfamily of sTNF receptors (sTNF-Rs); and
an adduct covalently linked to the receptor protein that increases the half-life of the composition in the subject, and the composition having decreased immunogenicity than the full-length or the truncated form of the receptor protein alone, or than a corresponding PEGylated form of the protein.

2. The composition according to claim 1, wherein the receptor protein is at least one selected from the TNF receptor superfamily consisting of: sTNF-RI, sTNF-RII, DR6, CD95, DcR3, DR3, Fn14, DR4, DR5, DcR1, DcR2, OPG, RANK, HVEM, LTβR, GITR, CD40, CD30, CD27, OX40, 41BB, NGFR, BCMA, TACI, BR3, XEDAR, EDAR, TROY, and RELT.

3. The composition according to claim 2, wherein the receptor protein is at least one selected from the group consisting of a p55 TNFα monomeric receptor (sTNF-RI) protein; a p75 TNFα monomeric receptor (sTNF-RII) protein; and the truncated form of the receptor protein comprising at least one of: domain 1 or a portion thereof, domain 2 or a portion thereof, domain 3 or a portion thereof, and domain 4 or a portion thereof.

4. The composition according to claim 1, wherein the composition is biodegradable in vivo in the subject.

5. The composition according to claim 1, wherein the composition is biodegradable by kidney enzymes of the subject.

6. The composition according to claim 1, wherein the adduct is a polypeptide containing proline and alanine, and/or serine (PAS polypeptide) or naturally occurring sugars containing heparosan molecules.

7. The composition according to claim 1, wherein the adduct is a linear polypeptide chain comprising at least one of natural amino acid residues or a combination of natural and unnatural amino acid residues.

8. The composition according to claim 1, wherein the adduct increases the half-life of the proteins at least about 10-fold.

9. The composition according to claim 1, wherein the adduct increases the half-life of the proteins by a factor of at least about 300-fold.

10. The composition according to claim 5, wherein the PAS polypeptide forms a monodisperse mixture as determined using mass spectroscopy.

11. The composition according to claim 1, wherein the adduct is covalently linked at the C-terminus of the receptor protein, or the N-terminus of the receptor protein.

12. The composition according to claim 1, wherein the adduct is a plurality of adducts, and a first adduct is covalently linked at the N-terminus and a second adduct is covalently linked at the C-terminus of the receptor protein.

13. The composition according to claim 1, wherein the adduct is covalently linked to the receptor protein at a position internal to the N-terminus and the C-terminus.

14. The composition according to claim 1, wherein the adduct is a plurality of adducts, and each of the plurality is covalently linked to a different domain of the receptor protein, and the domains are at least one selected from the group of domains of the full-length form of the receptor protein consisting of domains 1, 2, 3, and 4.

15. The composition according to claim 1, wherein the adduct further comprises at least one selected from the group of drugs consisting of: an anti-inflammatory drug, a steroidal drug, and a non-steroidal drug.

16. The composition according to claim 14, wherein the anti-inflammatory drug is methotrexate.

17. The composition according to claim 1, wherein the adduct is located at an immunogenic site of the receptor protein and masks the immunogenicity.

18. The composition according to claim 1, wherein the adduct is at least about 200 amino acid residues.

19. The composition according to claim 1, wherein the adduct is at least about 1200 amino acid residues.

20. The composition according to claim 1, wherein the half-life in vivo is at least about 25 hours, at least about 75 hours, at least about 125 hours, at least about 175 hours, at least about 225 hours, or at least about 275 hours.

21. The composition according to claim 1, wherein the receptor protein comprises at least one amino acid sequence selected from the group consisting of: SEQ ID NO: 3, SEQ ID NO: 4, SEQ ID NO: 5, SEQ ID NO: 6, SEQ ID NO: 7, and SEQ ID NO: 8.

22. The composition according to claim 1, wherein the composition further comprising an affinity tag for chromatographic purification.

23. A method of preventing or treating a subject for at least one of an inflammation or an autoimmune disease, the method comprising:
engineering a composition comprising a full-length or truncated form of a receptor protein that is a member of the superfamily of sTNF receptors (sTNF-Rs), and the receptor protein containing an adduct that increases the half-life of the protein in the subject, and the composition containing the adduct is less immunogenic than the receptor protein which is PEGylated; and
administering the composition to the subject.

24. The method according to claim 22, the method further comprising prior to administering, formulating the composition in a form that is effective for a prophylactic or a therapeutic use.

25. The method according to claim 22, the method further comprising prior to administering, genetically conjugating the adduct to the receptor protein.

26. The method according to claim 22, the method further comprising prior to administering, chemically conjugating the adduct to the receptor protein.

27. The method according to claim 22, the method further comprises prior to administering, increasing the half-life of the receptor protein by conjugating a PAS polypeptide or heparosan to the receptor protein.

28. The method according to claim 22, wherein prior to administering expressing the composition in prokaryotic cells or in eurkaryotic cells.

29. A composition for preventing or treating a subject for at least one of an inflammation, an autoimmune disease, and a metabolic disease, the composition comprising:
a truncated form of p55 TNFα monomeric receptor (sTNF-RI) protein, the truncated form of sTNF-RI protein comprising the amino acid sequence consisting of SEQ ID NO: 6; and
a PAS polypeptide covalently linked to the protein that increases the half-life of the composition in the subject, the PAS polypeptide having a length of at least about 600 amino acid residues.


SEQUENCE LISTING (9 sequences)

SEQ ID NO: 1 - protein, 1362 aa, Artificial Sequence
  The sequence was designed and synthesized
ATGGGCCTCTCCACCGTGCCTGACCTGCTGCTGCCGCTGGTGCTCCTGGAGCTGTTGGTGGGAATATACCCCTCAGGGGTTATTGGACTGGTCCCTCACCTAGGGGACAGGGAGAAGAGAGATAGTGTGTGTCCCCAAGGAAAATATATCCACCCTCAAAATAATTCGATTTGCTGTACCAAGTGCCACAAAGGAACCTACTTGTACAATGACTGTCCAGGCCCGGGGCAGGATACGGACTGCAGGGAGTGTGAGAGCGGCTCCTTCACCGCTTCAGAAAACCACCTCAGACACTGCCTCAGCTGCTCCAAATGCCGAAAGGAAATGGGTCAGGTGGAGATCTCTTCTTGCACAGTGGACCGGGACACCGTGTGTGGCTGCAGGAAGAACCAGTACCGGCATTATTGGAGTGAAAACCTTTTCCAGTGCTTCAATTGCAGCCTCTGCCTCAATGGGACCGTGCACCTCTCCTGCCAGGAGAAACAGAACACCGTGTGCACCTGCCATGCAGGTTTCTTTCTAAGAGAAAACGAGTGTGTCTCCTGTAGTAACTGTAAGAAAAGCCTGGAGTGCACGAAGTTGTGCCTACCCCAGATTGAGAATGTTAAGGGCACTGAGGACTCAGGCACCACAGTGCTGTTGCCCCTGGTCATTTTCTTTGGTCTTTGCCTTTTATCCCTCCTCTTCATTGGTTTAATGTATCGCTACCAACGGTGGAAGTCCAAGCTCTACTCCATTGTTTGTGGGAAATCGACACCTGAAAAAGAGGGGGAGCTTGAAGGAACTACTACTAAGCCCCTGGCCCCAAACCCAAGCTTCAGTCCCACTCCAGGCTTCACCCCCACCCTGGGCTTCAGTCCCGTGCCCAGTTCCACCTTCACCTCCAGCTCCACCTATACCCCCGGTGACTGTCCCAACTTTGCGGCTCCCCGCAGAGAGGTGGCACCACCCTATCAGGGGGCTGACCCCATCCTTGCGACAGCCCTCGCCTCCGACCCCATCCCCAACCCCCTTCAGAAGTGGGAGGACAGTGCCCACAAGCCACAGAGCCTAGACACTGATGACCCCGCGACGCTGTACGCCGTGGTGGAGAACGTGCCCCCGTTGCGCTGGAAGGAATTCGTGCGGCGCCTAGGGCTGAGCGACCACGAGATCGATCGGCTGGAGCTGCAGAACGGGCGCTGCCTGCGCGAGGCGCAATACAGCATGCTGGCGACCTGGAGGCGGCGCACCCGGCGCGAGGCCACGCTGGAGCTGCTGGGACGCGTGCTCCGCGACATGGACCTGCTGGGCTGCTTGGAGGACATCGAGGAGGCGCTTTGCGCCCCGCCGCTCCCGCCCGCGCCCAGTCTTCTCAGATGA

SEQ ID NO: 2 - protein, 315 aa, Artificial Sequence
  The sequence was designed and synthesized
GATAGTGTGTGTCCCCAAGGAAAATATATCCACCCTCAAAATAATTCGATTTGCTGTACCAAGTGCCACAAAGGAACCTACTTGTACAATGACTGTCCAGGCCCGGGGCAGGATACGGACTGCAGGGAGTGTGAGAGCGGCTCCTTCACCGCTTCAGAAAACCACCTCAGACACTGCCTCAGCTGCTCCAAATGCCGAAAGGAAATGGGTCAGGTGGAGATCTCTTCTTGCACAGTGGACCGGGACACCGTGTGTGGCTGCAGGAAGAACCAGTACCGGCATTATTGGAGTGAAAACCTTTTCCAGTGCTTCAAT

SEQ ID NO: 3 - protein, 453 aa, Artificial Sequence [sequence omitted]

SEQ ID NO: 4 - protein, 161 aa, Artificial Sequence
  The sequence was designed and synthesized
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 5 - protein, 127 aa, Artificial Sequence
  The sequence was designed and synthesized
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCT

SEQ ID NO: 6 - protein, 108 aa, Artificial Sequence
  The sequence was designed and synthesized
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 7 - protein, 100 aa, Artificial Sequence
  The sequence was designed and synthesized
YIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 8 - protein, 461 aa, Artificial Sequence
  The sequence was designed and synthesized
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 9 - protein, 20 aa, Artificial Sequence
  The sequence was designed and synthesized
ASPAAPAPASPAAPAPSAPA